(19) United States
(12) Patent Application Publication
(10) Pub. No.: US 2017/0079943 A1
(43) Pub. Date: Mar. 23, 2017

(54) PROTECTIVE METALLOTHIONEIN ANALOG COMPOUNDS, THEIR COMPOSITIONS AND USE THEREOF IN THE TREATMENT OF PATHOGENIC DISEASES

(71) Applicant: Albert Crum, Rhinebeck, NY (US)

(72) Inventor: Albert Crum, Rhinebeck, NY (US)

(21) Appl. No.: 15/367,762

(22) Filed: Dec. 2, 2016

Related U.S. Application Data
(63) Continuation of application No. 14/879,319, filed on Oct. 9, 2015.
(60) Provisional application No. 62/062,015, filed on Oct. 9, 2014.

(51) Int. Cl. A61K 31/198; A61K 33/04; A61K 45/06

(52) U.S. Cl. CPC A61K 31/198; A61K 45/06; A61K 33/04

(57) ABSTRACT

Embodiments of the present invention relate generally the use of certain compositions, e.g., compositions comprising a glutathione precursor and a selenium source, in the therapy of viral diseases and/or reducing the incidence of viral diseases. Related embodiments of the present invention relate to treatment and/or reducing the incidence of respiratory ailments caused by respiratory syncytial virus (RSV) or hemorrhagic fever (EHF) caused by Ebola viruses (EBV) or Marburg virus. Yet in other embodiments, the invention relates to reducing metal toxicity in a biological system, which involves contacting the biological system with a composition comprising a glutathione precursor and a selenium source, optionally together with a chelating agent, an antioxidant, a metallothioneine protein or a fragment of metallothioneine.

### FIELD OF THE INVENTION

Embodiments of the present invention relate generally to the use of certain metallothionein analogs, e.g., compositions comprising a glutathione precursor and a selenium source, as novel agents for the treatment of pathogenic diseases.

### GOVERNMENT FUNDING

No government funds were used in making the invention herein disclosed and claimed.

### BACKGROUND TO THE INVENTION

Metallothioneins (MT) belong to a family of cysteine-rich, low molecular weight (MW ranging from 500 to 14000 Da) proteins. They are localized to the membrane of the Golgi apparatus. MTs have the capacity to bind both physiological heavy metals (such as zinc, copper, selenium) and xenobiotic heavy metals (such as cadmium, lead, mercury, silver, arsenic) through the thiol group of its cysteine residues, which represents nearly the 30% of its amino acidic residues. They are thought to play a role in metal detoxification or in the metabolism and homeostasis of metals. MTS are present in a wide variety of eukaryotes including invertebrates, vertebrates, plants, and fungi. See, Sigel et al. “Metallothioneins and related chelators: Metal Ions in Life Sciences, Cambridge, England: Royal Society of Chemistry (ISBN 1-84755-899-2), which is incorporated by reference in parts pertinent thereto.

Since acute or chronic exposure to heavy metals such as lead, arsenic, mercury or cadmium is implicated in the ctiology of a variety of diseases and disorders involving neuromuscular, CNS, cardiovascular, and gastrointestinal effects, metallothioneins have been postulated to play a role in the prevention or alleviation of these conditions. However, a direct and distinct role of metallothioneins in the reduction of incidence and/or treatment of pathogenic diseases, e.g., viral diseases, is unknown.

It was also previously postulated that the aforementioned biological functions of metallothioneins, e.g., proper functioning of neuromuscular, CNS, cardiovascular, and gastrointestinal systems, were only accomplished with low molecular weight metallothionein proteins with a reference range of 500 to 14,000 daltons (Da). Moreover, synthetic derivatives and precursors of metallothionine were unknown, as most of the earlier work on this area focused on biological isoforms of metallothionine (e.g., MT-I and MT-II) and fragments thereof. See Hillman et al. (U.S. Pat. No. 5,955,428) and Berezin et al. (U.S. Pat. No. 8,618,060), the disclosures in which are incorporated by reference herein in their entirety. Ideally, the metallothioneine fragments described herein and in literature have similar or identical biological activity as the full-length proteins (e.g., ability to sequester metal ions).

Genetic delivery of metallothionine isoforms and fragments thereof presents numerous challenges, e.g., technical hurdles associated with the delivery of the gene precisely to target cells; and side effects, such as, infection (due to the vectors used in gene delivery) and tumor development (due to misplaced integration of the gene). Even when delivered properly, the biological metallothionine isoforms and fragments thereof are only located in the membrane of the Golgi apparatus and thus not cytosolically available.

Similarly, delivery of complex proteins of metallothionine isoforms is cumbersome, costly, difficult to manufacture in clinical grade and purity, and also face efficacy issues. In this context low molecular weight peptides, e.g., metallothionine fragments, are more bio-available.

Finally, although the biological role of metallothionine has been elucidated in literature, its utility is limited to chelation of metals from samples. For example, there is little, if any evidence to suggest use of metallothionines in the prevention or treatment of pathogenic diseases. Thus, there exists an urgent need for the treatment of and/or reduction of the incidence of pathogenic diseases, e.g., viral diseases, in subjects exposed to such pathogens. In the case of viral diseases where there is no current therapy, such as, Ebola virus disease (EVD) or Ebola hemorrhagic fever (EHF), there is increasing need for novel agents for the prophylaxis, therapy, chelation therapy and supportive therapy, and management of subjects afflicted with such diseases.

### SUMMARY OF THE INVENTION

### Rationale for the Use of Metallothioneine Analogs in Antiviral/Anticancer Therapy

Embodiments provided herein build upon the recognized role of a selected group of metalloproteins, particularly viral (v) and cellular (c) zinc finger proteins (ZFP) and iron containing proteins in cell proliferation, neovascularization, apoptosis, and viral infection. Along these lines the instant inventor envisioned that disruption of certain metalloproteins by novel pharmacological agents may serve to control and reduce the incidence of many viral and proliferative diseases. In this regard, embodiments provided herein relate to the potential therapeutic applications of ZFP disrupting agents, zinc chelators and iron chelators in the control of viral and/or proliferative diseases. Examples of such proliferative disorders include, but are not limited to, virally transformed cells and cancers relating thereto (e.g., Kaposi's sarcoma, Burkett's lymphoma, adult T-cell leukemia, Merkel cell carcinoma, papilloma-virus induced cancers of cervix, vulva, vagina, penis, anus, etc., and nasopharyngeal carcinoma, etc.).

Due to the central importance and essential functions of viral and cellular zinc-finger proteins, the literature on these topics is now rapidly expanding. Different aspects of ZFP functions, for example, in apoptosis induced by viruses, have been reviewed in recent years. Embodiments of the present invention thus relate to various zinc finger proteins of viruses and cellular zinc finger proteins induced by virus infection, including agents that inhibit their function, in an attempt to critically evaluate some basic biological consequences of manipulating zinc finger proteins.

### Conserved Relationship Between ZFP and Viral Replication

All viruses depend on their ability to infect cells and induce them to make more virus particles. If the virus is successful the cells almost invariably die in the process, and that process have been shown to be apoptosis in numerous instances. Other viruses can integrate its DNA in the cellular DNA and remain inactive for long periods. The nucleic acid genome of viruses is always surrounded by a protein shell, denoted capsid, which is composed of nucleocapsid proteins, and some viruses also have a lipid bilayer membrane, termed an envelope, which enclose the nucleocapsid proteins.

Viral ZFPs have been identified in at least two thirds of all viruses studied. See Fernandez-Pol et al, “Essential Viral and Cellular Zinc and Iron Containing Metalloproteins as Targets for Novel Antiviral and Anticancer Agents: Implications for Prevention and Therapy of Viral Disease and Cancer,” Anticancer Research vol. 21:931-958, 2001, which is incorporated by reference in parts pertinent thereto. Examples of families of viruses using metalloproteins such as ZFP, zinc ring proteins or transition metal ion-dependent enzymes for replication, packaging and virulence are Arenaviridae, Reoviridae, Rotaviridae, Retroviridae, Papillomavirinae, Influenza, Adenoviridae, Flaviviridae (Hepatitis C), Herpesviridae, Filoviridae (e.g., Ebola virus and Marburg virus), Pneumovirinae (e.g., RSV), Orthomyxoviridae (Influenza viruses), etc. Viral ZFP are structural virion proteins essential for viral replication and packaging of the virus inside infected cells. Deletion of zinc finger domains in specific vZFP is lethal to the virus. Since the zinc finger domains of vZFP are essential for viral survival functions, they are conserved throughout evolution and there are no known mutants of the vZFP domain(s). Because the viral zinc finger domain(s) represent indispensable site (s) on the vZFP that can be attacked by one or multiple drugs, vZFP are ideal and primary drug targets for the next generation of antiviral agents. Representative examples of viruses which rely on metalloproteins and specifically zinc-binding proteins such as ZFP, for replication and virulence are characterized below:

Papilloma virus infection results in a number of proliferative diseases in humans including warts induced by type 4 human papilloma virus (common warts). Moreover, papilloma virus can cause plantar ulcers as well as plantar warts. Human papilloma virus infection of the uterine cervix is the most common of all sexually transmitted diseases. Commonly know as genital warts, this wide spread virus infection is a serious disease that potentially can develop into cervical cancer. Since the virus is permanently present in cells, infection recurs in a significant percentage of patients.

Condylomata acuninata, also denoted genital warts, are benign epithelial growths that occur in the genital and perianal areas and caused by a number of human papilloma viruses (HPV) including types 6, 11 and 54. These are low risk viruses which rarely progress to malignancy. However, high risk viruses such as HPV-16 and HPV-18 are associated with cervical intraepithelial cancer. The actions of HPV are mediated by specific viral-encoded proteins which interact and/or modulate cellular DNA and proteins to produce abnormal growth and differentiation of cells. Two proteins of the HPV viral genome, E6 and E7, are well conserved among anogenital HPV's and both contribute to the uncontrolled proliferation of basal cells characteristics of the lesions. The E7 oncoprotein is a multi-functional protein with transcriptional modulatory and cellular transforming properties. The E7 oncoprotein is a zinc finger protein.

Herpes viruses are highly disseminated in nature. Herpes viruses vary greatly in their biological properties and the clinical manifestations of diseases they cause. In humans eight herpes viruses have been isolated to date: 1) herpes simplex virus 1 (HSV-1), herpes simplex virus 2 (H1SV-2), cytomegalovirus (HCMV), varicella-zoster virus (VZV), Epstein-Barr virus (EBV), human herpesvirus 6 and 7 (HHSV6 and HHSV-7). More recently the existence of HHV8 as a causative agent of Kaposi sarcoma has been documented. The known herpesviruses share two significant biological properties relevant to this invention: 1) all herpesviruses specify a large array of enzymes involved in nucleic acid metabolism, including ribonucleotide reductase, an iron containing enzyme; and 2) they possess major zinc finger DNA-binding proteins required for DNA replication.

Retrovirus virions contain a diploid genome consisting of an RNA complex formed by the association of two identical unspliced viral RNA molecules. In mature virions, RNA molecules are tightly bound to viral zinc finger proteins, denoted nucleocapsid proteins (Ncps). Retroviral Ncp is produced after the gag gene product (Pr55gag), has been processed by the viral protease. The Ncps are highly conserved in all known retroviruses. Point mutation of the cysteine and histidine residues of the zinc finger domain of NCp7 results in a radical reduction of genomic RNA packaging, and this results in a drastic decrease in viral infectivity. Further studies indicate that NCp7 plays a key role in several other steps of the viral life cycle.

The human immunodeficiency virus (HIV) encodes several regulatory proteins that are not present in other retroviruses. The nucleocapsid p7 protein of HIV has been targeted by the inventor and other investigators for treatment of UW viral infections. The p7 protein is required for the correct assembly of viral RNA in newly formed virus particles. The p7 protein contains two zinc fingers that are critical for the recognition and packaging of the viral RNA. Thus, agents that effectively attack the two zinc finger domains of the HIV virus nucleocapsid p7 protein inside infected cells will decrease the overall number of viral infective particles.

The influenza virus is not integrated with DNA and thus may be vulnerable to attack by the specific antiviral agents of this invention. The influenza viruses are dependent upon viral Zn²⁺ metalloproteases for specific viral functions. Processing of critical proteins of influenza virus is mediated by virus-encoded Zn²⁺ metalloproteases. It is of interest for this invention that the most abundant virion protein and a type specific antigen of influenza viruses, the M1 protein, is a zinc finger protein. Furthermore, this protein is involved in packaging of the influenza virus. Thus, inhibition of influenza virus Zn²⁺ metalloproteinases and/or zinc finger protein M1 by the agents of this invention presents an opportunity for controlling the progression of influenza virus infection.

Human respiratory syncytial virus (RSV), which is closely related to the flu virus, is a virus that causes respiratory tract infections. It is a major cause of lower respiratory tract infections and hospital visits during infancy and childhood. Research has established that the RSV virus and certain other viruses require elemental zinc in order to replicate and proliferate. See, Esperante et al. (“Fine Modulation of the Respiratory Syncytial Virus M2-1 Protein Quaternary Structure by Reversible Zinc Removal from its Cys3-His1 Motif,” Biochemistry, 52 (39), pp 6779-6789, 2013), which is incorporated by reference herein.

The poxviridae is a large family of complex DNA viruses that replicate in the cytoplasm of vertebrate and invertebrate cells. The most notorious virus of this family is the variola virus that causes smallpox. Infectious poxvirus particles contain a complex transcription system. A large number of virus-encoded enzymes and factors are packaged in the virus particle. For example, RNA polymerase, a zinc requiring enzyme, is involved in early transcription. Furthermore, both the small catalytic subunit and the large regulatory subunit of ribonucleotide reductase are virus-encoded proteins and closely resemble their eukaryotic counterparts both structurally (80% homology) and functionally. The synthesis of ribonucleotide reductase, is induced rapidly after vaccinia virus infection. Catalytic activity of the small subunit is inhibited by hydroxyurea. Furthermore, some of the early viral and cellular transcription factors utilized by the smallpox virus are zinc finger proteins.

Filoviruses, which cause deadly hemorrhagic fevers, are a large group of viruses that have non-segmented negative-strand (NNS) RNA as their genomes. The two main types are the Marburg and the Ebola virus. The nucleoproteins of these viruses interact with the linear RNA genome and also with cellular and ribosomal zinc finger proteins to perform specific viral functions. Thus, filoviruses are susceptible to inhibition by the agents of this invention.

Ebola virus (EBV, formerly designated Zaire ebolavirus) and its closely-related Marburg virus, which fall within the genus Ebolavirus, are also known to utilize zinc-binding proteins for replication. These viruses are known to cause a severe and often fatal hemorrhagic fever in humans and other mammals, known as Ebola virus disease (EVD). Ebola virus has caused the majority of human deaths from EVD, and is the cause of the 2013-2015 Ebola virus epidemic in West Africa, which has resulted in at least 28,424 suspected cases and 11,311 confirmed deaths. It has been scientifically established that the Ebola virus requires zinc for its replication and proliferation. Without elemental zinc, the Ebola virus cannot survive. See, for example, Modrof et al. (“Ebola Virus Transcription Activator VP30 is a Zinc-Binding Protein,” Journal of Virology, March 2003, p. 3334-3338); Enterlein et al. (“Rescue of Recombinant Marburg Virus from cDNA is Dependent on Nucleocapsid Protein VP30,” Journal of Virology, January 2006, p. 1036-1043); John et al. (“Ebola Virus VP30 Is an RNA Binding Protein,” Journal of Virology, 81(17):8967-8976, 2007); Hartlieb et al. (“Oligomerization of Ebola virus VP 30 is essential for viral transcription and can be inhibited by a synthetic peptide,” Journal of Biological Chemistry, 278(43), 40830-40836, 2003) and Esperante et al. (“Fine Modulation of the Respiratory Syncytial Virus M2-1 Protein Quaternary Structure by Reversible Zinc Removal from its Cys3-His1 Motif,” Biochemistry 2013, 52, 6779-6789), the disclosures in which are incorporated by reference herein in their entirety.

There are numerous examples of families of viruses that utilize zinc finger proteins, zinc ring proteins and/or transition metal ion-dependent enzymes for specific viral functions. These viral proteins play an essential role in the structure, replication and/or virulence of viruses such as Reoviruses, Rotaviruses, Hepatitis C viruses as well as numerous other viruses.

### Targeting of ZFP for Therapy

The National Cancer Institute has identified ZFP as the next target for antiviral drugs (USA Federal Register, 60, No. 154, 1995). Several laboratories are evaluating new antiviral drugs targeted to modify ZFP. These products are targeted towards modification of the amino acid cysteine, which is the binding site for zinc in zinc finger proteins. The present inventor have identified that the cysteine residue of the glutathione molecule, which is synthesized via reconstitution of the precursor components, e.g., glycine, cysteine (as cystine) and glutamate source (for example, glutamine or glutamic acid) confers inhibition of the replication of viruses that rely on such Zn²⁺-binding proteins. Examples of such viruses include, but are not limited to, Ebola viruses (EBV), respiratory syncytial virus (RSV), HIV, HPV, and HSV.

It has been known for many years that the structural and biological properties of viruses can be altered by chelating agents. For example, treatment of rotaviruses with chelating agents such as ethylenediaminetetraacetic acid (EDTA) (10 mM) results in a single-shelled, double-layered, non-infectious viral particles. Moreover, in vitro exposure of various retroviruses to the chelating agents such as EDTA or ethylene glycol tetraacetic acid (EGTA) in millimolar concentrations results in partial disintegration of viral membranes. Thus, disintegration and degradation of retroviruses and rotaviruses can be accomplished by chelating agents.

Similarly, Muller et al. (“Inhibition of filovirus replication by the zinc finger antiviral protein,” Journal of Virology, 81(5):2391-400, 2007) studied a role of zinc finger antiviral protein (ZAP) against Ebola virus (EBOV) and Marburg virus (MARV). Antiviral effect was observed in cells expressing the N-terminal part of ZAP fused to the product of the zeocin resistance gene (NZAP-Zeo) as well as cells inducibly expressing full-length ZAP. EBOV was inhibited by up to 4 log units, whereas MARV was inhibited between 1 to 2 log units. Transient expression of ZAP decreased the activity of an EBOV replicon system by up to 95%. This inhibitory effect could be partially compensated for by overexpression of L protein. In conclusion, Muller states that the data demonstrate that ZAP exhibits antiviral activity against filoviruses.

Other zinc-binding proteins involved in viral infectivity include, for example, members of the ADAM family of the metalloproteinases. For example, Dolnick et al. (“Ectodomain shedding of the glycoprotein GP of Ebola virus,” The EMBO Journal: 23, 2175-2184, 2004) show that tumor necrosis factor α-converting enzyme (TACE), a member of the ADAM family of zinc-dependent metalloproteases, is involved in the shedding of surface glycoproteins in Ebola viruses. Dolnick further shows that virus-encoded surface glycoproteins are substrates for ADAMs, which cleave them to release them in the blood of virus-infected animals and TACE may play an important role in the pathogenesis of infection by efficiently blocking the activity of virus-neutralizing antibodies. Moreover, inhibitors of zinc-dependent metalloproteinases were shown to inhibit glycoprotein shedding in a concentration-dependent manner. The inhibitory effects were observed with the hydroxamic acid-based inhibitors: BB2516 used at a concentration of 0.5 mM, and GM6001 and MMP-8 inhibitor I used at a concentration of 5 mM. Other inhibitors, such as MMP-3 inhibitor II, CGS-27023A, and TAPI-I, reduced GP shedding at higher concentrations (25-50 mM).

### Use of Chelating Agents to Inhibit Viral Replication

There are several chelating agents that eject the coordinately bound zinc atom from HIV zinc finger proteins. For example, Otzuka et al reported that novel zinc chelators inhibit the DNA-binding activity of zinc finger proteins of HIV. In addition, The Tat trans-activator, is a small protein of 75-130 amino acids, which may form a zinc-finger domain. Since HIV-1 lacking Tat replicates poorly and does not cause cytopathic effects, approaches to interfere with Tat may be useful in treating AIDS. The cysteine-rich domain of Tat binds divalent cations, either two Cd2+ or two Zn²⁺ atoms. Whether the cysteine-rich residues form a Zn²⁺ finger or lattice binding pockets for divalent cations is unknown. The pol gene also has a zinc finger amino acid sequence suggesting that chelation chemotherapy may have a role in the treatment of AIDS.

Other research points to the use of competitive inhibition (using peptides that bind to Zn²⁺) as anti-viral agents. See, Hartlieb et al., Journal of Biological Chemistry, 278(43), 40830-40836, 2003.

At least three efficient approaches may be used to design novel classes of inhibitors of viral ZFP activity that directly attack vZFP: 1) disruption of the zinc finger domain by modification of the cysteine residues which are the binding sites for Zn²⁺ in the vZFP, resulting in the ejection of zinc ion; 2) removal of the zinc from the zinc finger moiety by specific chelating agents, which results in inactivation of the vZFP; and 3) specific chelating agents that form a ternary complex at the site of zinc binding on vZFP, resulting in inhibition of the DNA or RNA binding activity of vZFP. Since these antiviral agents attack highly conserved structures in the virus they may circumvent the emergence of drug resistant mutants. Furthermore, the basic mechanisms of action of the novel antivirals (1 through 3, above) may be enhanced in viral disease if the antiviral agents which directly attack metalloproteins of the virus simultaneously attack cellular metalloproteins implicated in the pathogenesis of viral disease. Hence, the novel antivirals may also prove to be effective against cellular zinc finger-containing proteins such as ribosomal ZFP and heat shock proteins which are involved in viral infection. These cellular proteins are induced by the virus for specific viral functions such as replication, propagation, or as an inflammatory response of the cells to the virus.

The specificity of these agents may be due to cellular specificity, in which virally infected cells express cellular and viral ZFPs that are not expressed by normal uninfected cells in their basal or proliferative state. Another primary mode of action of these agents could be receptor specificity, in which vZFP act as receptors for specific zinc ejecting agents, or specific chelating agents which bind to vZFP and form an inactive ternary complex consisting of vZFP-Zn-chelating agent. Thus, vZFP may act as receptors for new agents that can form ternary complexes with vZFP.

### Use of Metallothionine Analogs

Embodiments of the present invention provide a solution to the aforementioned problems associated with the delivery and/or use of biological metallothionines. In one embodiment, there is provided a metallothionine analog comprising a glutathione (GSH) precursor, optionally together with a selenium source. The glutathione precursor comprises (a) L-glycine; (b) L-cystine; and (c) a glutamate source (e.g., glutamine or glutamate), which precursor confers intracellular synthesis of glutathione. See Crum et al. (US patent app. pub. No. 2012-0029082), which is incorporated by reference herein in its entirety. See also U.S. Reissue Pat. Nos. 39,734 and 42,645, which are incorporated by reference herein. Accordingly, embodiments of the instant invention relate to the use of glutathione formed by the regulated physiological process pathway (trademarked as VITAMIN GSH-S®) as a protective metallothioneine analog compound.

In synthesizing glutathione in the body, cysteine, a thiol amino acid is required. Background research suggests that oral administration of glutathione itself would be ineffective and that prodrugs or precursor therapy would be necessary. Cysteine, or a more bioavailable precursor of cysteine, N-acetyl cysteine (NAC), has been suggested as candidates for precursor therapy. While cysteine and NAC are both, themselves, antioxidants, their presence competes with glutathione for resources in certain reducing (GSH recycling) pathways. Since glutathione is a specific substrate for many reducing pathways, the loading of a host with cysteine or NAC may result in less efficient utilization or recycling of glutathione. Thus, cysteine and NAC are not ideal GSH prodrugs. Thus, while GSH may be degraded, and non-physiologically transported as amino acids, there is a physiological barrier to the importation of intact glutathione. None of the former methods provide a reliable and safe means for increasing intracellular GSH levels.

The compositions and methods of the embodiments described herein therefore provide an improvement over art-known methods for increasing glutathione levels, including importation of intact glutathione molecule into the cytosol using liposome and the like. However, whole glutathione importation into the cell negates the physiologically-perfected synthesis pathway's enzymatic process.

In contrast to the aforementioned suggestions using cysteine or NAC as prodrugs for enhancing cellular GSH levels, embodiments of the present invention relate to alternative methods for elevating levels of physiologically synthesized glutathione and using the glutathione to combat many viral and other pathogenic diseases. In such embodiments, the target system (e.g., cell, tissue, organ or organism) is provided with the components of glutathione (e.g., (a) L-glycine; (b) L-cystine; (c) a glutamate source, e.g., glutamine or glutamate) and optionally the selenium source. The physiologically synthesized glutathione can function as a metallothioneine by modulating the optimal reference range for biochemical elemental metals, such as zinc and copper. The metallothioneine role can also protect the host from the toxicity of heavy metals (cadmium, lead, silver, arsenic, et al). Unlike biological metallothioneine and fragments thereof, the sulfhydryl activity and function of the physiologically synthesized GSH is not limited to molecular weight proteins of 500 to 14,000 daltons, which are located in the membrane of the Golgi apparatus.

In embodiments described herein, the sulfhydryl of a composition previously characterized in RE42,645E can serve a protective function for the host by protecting the body from viral challenges that require elemental metals in order to replicate and proliferate. In the case of Ebola viruses, e.g. causative agents of the Ebola virus disease (EVD) or Ebola hemorrhagic fever (EHF), the virus requires zinc (Zn²⁺). In other viruses such as hepatitis C virus (HCV), the causative agent of human hepatitis, replication is enhanced in the presence of iron (Fe²⁺). The compositions of the instant invention, as ion chelators and/or sequestering agents, reduce the infectivity of the pathogenic agents.

Role of Iron-Binding Proteins in Viral Replication

Growing literature implicates a role of iron-binding proteins in the replication and infection of viruses such as herpes simplex (HIV-1 and HIV-2), Epstein-Barr virus (EBV), varicella-zoster virus (VZV), pseudorabies virus (PRV), and equine herpesvirus type I (EHV-1). Ribonucleotide reductase (RR), which is formed by the association of two non-identical subunits (R1 and R2), catalyzes the reduction of ribonucleoside diphosphates to their 2′-deoxy derivatives which is a key intermediate in DNA biosynthesis. There is increasing evidence supporting the essentiality of ribonucleotide reductase (RR) in viral replication. Numerous organisms, including herpes viruses, bacteria, and mammals, encode ribonucleotide reductases the share a number of common characteristics. Two important characteristics of RR are the presence of a stable tyrosyl free radical and the dependency of Fe (III) for catalytic activity. The smaller (R2) subunit contains the iron and tyrosyl radical and the larger (R1) contains thiols which are redox active and provide the hydrogen for nucleotide reduction. The association of R1 and R2 are required for catalytic activity.

Thus, a potential approach for antiviral therapy would be the utilization of peptides that can inhibit enzymatic activity by preventing the association of R1 and R2 subunits. However, since iron is required for catalytic activity a potential, less specific, strategy for antiviral therapy are iron chelating agents, which would deplete iron from the cells, and may have a significant activity against herpes viruses. In 1998 picolinic acid was tested at 3 to 1.5 mM on cultured Human Foreskin (HF) cells infected with HSV-2-strain G and it was found to cause apoptosis of HF infected cells. The specificity of the iron chelators may be cellular specificity rather than viral specificity: infected cells enter apoptosis versus non-infected cells which remain unaffected. See, Romeo et al. (“Intracellular chelation of iron by bipyridyl inhibits DNA virus replication: ribonucleotide reductase maturation as a probe of intracellular iron pools,” Journal of Biological Chemistry, 276(26):24301-8, 2001), which is incorporated by reference herein.

It is relevant to mention that cellular RR is not only an important virulence factor for herpes viruses, but that cellular RR is also involved in the virulence of HIV. It has been suggested that the inhibition of RR with agents such as hydroxyurea could have a possible application in the treatment of AIDS. Giacca et al have found synergistic antiviral actions of ribonucleotide reductase inhibitors and 3′-azido-3′-deoxythymidine on HIV-1. RR inhibitors reduce the cellular supply of DNA precursors (dNTP) by interfering with their de novo synthesis. A secondary effect is the stimulation of the uptake and phosphorylation of extracellular deoxynucleosides, including their analogs such as 3′-azidothymidine (AZT). Both effects are important to HIV replication, which requires dNTP and is impaired by the triphosphate of AZT. A clear synergism between AZT and RR inhibitors was observed at nontoxic doses.

In vitro studies have shown that glutathione in free form binds iron, particularly Fe²⁺, with high affinity. See, Khan et al. (“Kinetic and spectrophotometric studies of binding of iron(III) by glutathione,” Canadian Journal of Chemistry, 54(20): 3192-3199, 1976), which is incorporated by reference herein in parts pertinent thereto. In accordance therewith, embodiments of the instant invention provide methods of inhibiting pathogenesis of bacterial, viral, or fungal diseases in which iron-binding proteins are implicated in the replication and/or propagation of the pathogenic agents.

### Role of Metal-Binding Proteins in Carcinogenesis and Anti-Apoptotic Pathways

Transition metal ions at physiological concentrations, such as chromium, zinc, iron, cobalt, and copper, are essential elements for biological functions; however in higher quantities they are toxic (Fernandez-Pol, et al, 2001). Evidence indicates that elevated levels of iron contribute to carcinogenesis. Two main factors are important in iron induced oncogenesis: 1) The capacity of iron to generate highly reactive free radicals which damage DNA; and 2) the increase iron requirement by rapidly proliferating transformed cells, which is required for DNA replication (ribonucleotide reductase; RR) and energy production (within the mitochondrial in key enzymes of the redox systems of the respiratory chain). Studies with iron chelating agents such as picolinic acid and desferoxamine have contributed significantly to the understanding of differential mechanisms of growth regulation in normal and transformed cells (Femandez-Pol et al, 2001, supra). It is known that iron induces mutagenesis and/or carcinogenesis, but the detail mechanism of iron-induced oncogenesis is unknown.

Initial in vitro studies have demonstrated the ability of cobalt and cadmium to structurally reconstitute the zinc finger domains in an active form. In contrast, nickel and copper bind to zinc finger proteins, but are unable to restore the DNA binding capacity. These studies suggest that heavy metal incorporation into zinc finger may be important in metal-induced toxicity. Recently, it has been found that an iron-substituted zinc finger may generate free radicals that damage DNA and potentially induced carcinogenesis. Further research has shown that human metallopanstimulin (MPS-1)/S27 ribosomal protein is a ubiquitous 9.4-kDa multifunctional “zinc finger” protein which is expressed at high levels in a wide variety of cultured proliferating cells and tumor tissues.

The human MPS-1 gene and its relationship to human cancer cell growth has been described in literature. See Fernandez-Pol wt. al., “Transcriptional regulation of proto-oncogene expression by epidermal growth factor, transforming growth factor beta-1, and triiodothyronine in MDA-468 cells”, Journal of Biological Chemistry, 264:4151-4156, 1989. Since that time, research has consistently demonstrated that both MPS-1 mRNA and protein are involved in cancer cell growth as demonstrated by increased levels of MPS-1 mRNA and protein found in numerous pathological tissue specimens obtained from various types of human cancers, such as prostate, breast, lung, colon, endometrium, uterine cervix, vulva, and melanoma. These results indicate that the MPS-1 antigen is a ubiquitous tumor marker that may be useful in detection and prognosis of various types of malignant neoplastic conditions. The results of other experiments indicate that MPS-1 is involved in protein synthesis, repair of damaged DNA, digestion of mutated mRNA, anti-apoptosis and rapid cell proliferation. Thus, the information available indicate that MPS-1 is a multifunctional S27 ribosomal protein relevant to numerous oncogenic processes that can be used as a ubiquitous tumor marker in various clinical assays. More recently, MPS-1/S27 ribosomal protein has been shown to be increased in virus infected cells, in parasites such as Toxoplasmosis and Malaria, in yeast proliferative capacity, and in macrophage activation in human melanomas NCBI, National Cancer Institute Data Bank; Fernandez-Pol, 2001).

It is important to note at this point that there are many reports indicating a connection between overexpression of some genes encoding ribosomal proteins and cancer. There is evidence that a number of other ribosomal proteins have additional functions separated from both the ribosome and protein synthesis. Zinc finger motifs are characteristics of numerous ribosomal proteins, allowing them to bind to nucleic acids. This binding ability offers a potential mechanism for ribosomal proteins to interfere in both transcriptional and translational mechanisms. For example, the rat ribosomal protein S3a is identical to the product of the rat Fte-1 gene that encodes the v-fos transformation effector. S3a is involved in the initiation of protein synthesis and is also related to proteins involved in the regulation of growth and the cell cycle. Rat ribosomal protein L10 is homologous to the Jun-binding protein and to a putative Wilm's tumor suppressor. Taken together, the findings of ribosomal proteins with oncogenic, tumor suppressor, or cell cycle functions, indicates extraribosomal functions of certain ribosomal proteins related to oncogenesis.

The involvement of zinc fingers in protein-protein interactions extends beyond the control of gene expression. In numerous proteins the zinc finger domains have been implicated in mediating homodimerization or heterodimerization (Fernandez-Pol et al, 2001, supra). Prokaryotes and eukaryotes express numerous heat shock proteins (HSP) in response to stress, including heat shock, exposure to heavy metals, hormones and viral infections.

The stress response that include numerous forms of physiological and pathological stress is involved in viral infection. A prominent feature of this response is the synthesis of a discrete set of zinc finger proteins, known as the heat shock proteins, which at present are denoted molecular chaperons. During infection by certain viruses, heat shock proteins act as intracellular detectors that recognize mis-folded proteins. Researchers have found that certain DNA viruses are able to activate heat shock proteins. For example, the Hsp70 (DnaK) is induced by adenovirus, herpes virus, cytomegalovirus, and other viruses. Furthermore, DnaJ, a heat shock protein that functions in the control of protein folding within the cell, contains two CCCC zinc finger motifs, defined by the J domain, which is essential for stimulation of the Hsp70 ATPase activity. Thus, the results indicate that there is a relationship between the stress response and the cytopathic effects of certain viruses such as herpes viruses, poxviruses, and hepatitis C viruses. Since Hsp70 has a protective role in inflammation, infection, and regulatory roles in cytokine biosynthesis, it has been postulated to play a vital role in viral replication. In accordance with the embodiments of the present invention, agents that can modify the zinc finger heat shock proteins are useful in controlling the viral replication.

### Apoptosis

A recent review summarizes the evidence that apoptosis is modulated by intracellular excess or deficiency of Zn²⁺ and presents some mechanism by which Zn²⁺ may control apoptosis (Fernandez-Pol, et al, 2001). The major conclusions are: 1) zinc deficiency, resulting from dietary deprivation or exposure of cultured cells to membrane-permeable Zn²⁺ chelators induces apoptosis; 2) zinc supplementation with Zn²⁺ to the media of cell cultures, can prevent apoptosis; and 3) an intracellular pool of chelatable Zn+ plays a critical role in apoptosis, possibly by modulating the activity of endonucleases. See, Femandez-Pol et. al., supra.

There is evidence that apoptosis is modulated by intracellular excess or deficiency of Zn²⁺. Fragmentation of DNA and cytolysis are inhibited in certain systems when Zn²⁺ (0.8 mM) is added to the culture medium, It is interesting to note that Ca²⁺/Mg²⁺-dependent endonuclease activity in isolated nuclei was inhibited when Zn²⁺ was added to the medium. These studies are consistent with the hypothesis that Zn²⁺ prevents apoptosis by blocking the activation or inhibiting the activity of Ca²⁺/Mg²⁺-dependent endonuclease. Numerous reports have shown that depletion of intracellular Zn²⁺ by chelation can trigger apoptosis in virally transformed cells. For example, when leukemia cells were exposed to 1,10-phenanthroline, a Zn²⁺/Fe²⁺ chelator, DNA fragmentation and cell death occurred, unless the chelator was neutralized by a transition metal ion added to the medium Similarly, picolinic acid (PA) a Zn²⁺/Fe²⁺ chelator, induces apoptosis in many cells, including leukemia cells by chelating a pool of intracellular Zn²⁺/Fe²⁺, since influx of Zn²⁺/Fe²⁺ prevented apoptosis in the presence of PA, while chelation of Zn²⁺/Fe²⁺ induced apoptosis.

Because Zn²⁺ plays a role in many cellular functions, and because it is a structural component of zinc finger proteins that are essential in cell replication, there are many sites in the apoptotic pathway that can be potentially modulate by zinc and zinc chelators. A number of investigators have shown that apoptosis can be induced if the intracellular level of Zn²⁺ are reduced using chelators. For example, N,N,N′,N′-tetrakis-2-pyridyl methyl-ethylene diamine (TPEN) added to cultured cells induces apoptosis. These experiments add additional support to the hypothesis that changes in intra- and extracellular zinc can modulate apoptosis. However, none of these chelators are specific for zinc, in fact, some of them are more specific for iron, and they may have chelated a variety of transition metals. Nevertheless, these studies indicate that zinc plays a complex role in a dose and time-dependent manner in apoptosis.

Viruses relevant to human disease such as Smallpox, Ebola virus, Marburg virus, Lassa virus, Papillomavirus, Herpes virus, and Retroviruses, including the AIDS virus, are all capable of inducing apoptosis. Viruses encode genes that both stimulate and suppress apoptotic cell death. These viral proteins interact with cellular pro-apoptotic (death factors) and anti-apoptotic (survival factors). Viral (v) and cellular (c) Zinc finger proteins (ZFP) are involved in apoptotic cell death. A pool of chelatable intracellular Zn²⁺ plays a critical role in viral and cellular apoptosis, possibly by modulating ZFP structure. In virally transformed cells, apoptosis can be induced by intracellular deficiency of Zn²⁺ while normal non-infected cells remain unaffected.

Research has shown that modulation of both v-ZFP and c-ZFP by a class of novel Zn²⁺/Fe²⁺ chelating, broad-spectrum antiviral agents may form ternary complexes with the zinc atoms contained in ZFP (42-60). In numerous experiments, research indicates that these wide-spectrum antiviral agents block viral replication and induced apoptosis in virally transformed cells in culture. These agents also interfere with abnormally expressed c-ZFP produced by spontaneously or radiation transformed cells in culture. Thus, these studies provide evidence for a close correlation between interference with ZFP of both viral and cellular origins and apoptosis in transformed but not in normal cells.

The methods of the invention find utility in the control or treatment of a variety of viruses and viral diseases, such as HIV, polio, human coxsackie, SARS, rabies, human parainfluenza, measles, human respiratory syncytial, and human hepatitis, Dengue, West Nile and Ebola. The aforementioned compositions may also be effective against malarial Plasmodium falciparum and Leishmania donovani parasites.

### EMBODIMENTS OF THE INVENTION

Accordingly, embodiments of the instant invention provide means for increasing the intracellular glutathione, can be effective competitively and physiologically extracting the metals and the co-factors (e.g., zinc) necessary for the propagation of viruses such as the Ebola virus. Without said elemental zinc, the virus cannot replicate, proliferate or survive.

In vitro chemical analyses have revealed that GSH is capable of binding to Zn²⁺ and Ni²⁺ with high affinity. See, Krezel et al. (“Studies of Zinc(II) and Nickel(II) complexes of GSH, GSSG and their analogs shed more light on their biological relevance,” Bioinorganic Chemistry & Applications; 2(3-4): 293-305, 2004), which is incorporated by reference herein in parts pertinent thereto. GSH is also capable of binding and thus sequestering Fe³⁺ ions. See Khan et al., Canadian Journal of Chemistry, 54(20): 3192-3199, 1976.

Although free glutathione might have sequestration capability in cell-free systems such as those described in Krezel and Khan, a variety of challenges are imposed in biological systems. For example, transition metals are not present in “free” states but rather bound to proteins in the form of complexes. Thus, glutathione is in direct competition with these proteins, e.g., ZFP or RR. Secondly, cellular absorption of glutathione is inefficient and thus intracellular glutathione levels are not appreciably increased by providing cells with free glutathione. Also, in the case of in vivo supplementation via the oral route, provision of free GSH is ineffective as the antioxidant is broken down in the gastrointestinal system of animals. Recognizing these and other limitations, the inventor of the instant application have contemplated novel ways to provide and ameliorate intracellular levels of glutathione. In accordance with the present invention, embodiments described herein provide compositions and means for using the physiological glutathione synthesis pathway for introducing intact glutathione into the cell and replenishing the cytosolic and other cellular compartments with glutathione. Herein, a distinction is drawn in this invention between using the step-by-step physiological synthesis of glutathione pathway as distinguished from other methods that would bypass the step-by-step physiological synthesis of glutathione. If the step-by-step pathway is bypassed, the bypassing process can eventually result in a weakened immune system and thus be counter-productive, by throwing the vital substrate-specific enzymes, which catalyze each step of the synthesis, into the vestigiality of disuse. In addition, if the physiological step-by-step synthesis pathway is avoided by the importation of intact glutathione, then the physiological regulatory feedback and shut-down mechanisms can be thrown into dysregulation. The biomarker of glutathione quantification would be lost, and its physiological regulation would become an uncertainty. The compositions and methods for increasing intracellular glutathione levels described herein avoid many of the aforementioned issues.

In protecting the host, the sulfhydryl moiety of physiologically synthesized glutathione competitively and effectively conjugates with the elemental metal such as zinc and copper and deprives the pathogenic virus of those metals, which the pathogenic viruses need to replicate and proliferate.

Accordingly, in an embodiment of the instant invention, the sulfhydryl group (—SH group) of the physiological cytosolic glutathione, if not compromised by the risk of vestigiality, has the biochemical and physiological efficiency to outmaneuver a pathologic virus such as Ebola in order to deprive that pathogen of zinc which viruses such as Ebola virus need to foster budding and survival. If glutathione is not physiologically synthesized, e.g., if the glutathione is imported as an intact molecule into the cytosol avoiding the step-by-step synthesis process, such a procedure can eventually weaken the immune system and thus fail to achieve the goal of glutathione therapy.

In accordance with the instant invention, embodiments described herein relate to increasing intracellular GSH levels by providing the individual components of glutathione, e.g., glycine, glutamate source (glutamine or glutamate) and cystine (a source of cysteine) optionally together with a selenium source. In this context, one skilled in the art understands that L-cystine is a metabolite amino acid in the catabolism of protein. It is found in certain protein foods, such as lean beef, clams, veal, turkey, chicken, fish, crabs, lobster, et al. L-cystine is a compound of two amino acids, L-cysteine and L-cysteine, which have auto-oxidized into a unity via a disulfide bond which unites these two L-cysteines into the new chemical molecule. L-cystine has radically different properties from the L-cysteine molecules from which it is formed. L-cystine can also be anabolized from L-methionine. It has a vital role in the metabolism of Vitamin B6. It was previously thought that because L-cystine is relatively stable, by virtue of its disulfide bond, that it was inactive, effete, oxidized or “used-up.”See, Emory University Public Press Release Apr. 4, 2011 “Measuring oxidative stress can predict risk of atrial fibrillation.”

In contrast to solo cysteine, which has little bodily physiological and biochemical functions, L-cystine, in addition to the above, is vital to the formation of insulin, sperm cells, skeletal muscle, connective tissues, hair and certain enzymes. Further, the disulfide bond serves many vital bodily biochemical and physiological functions (see list below). The use and role of the auto-oxidation in L-cystine is an evolutionary adaptation of major significance. However, the scientific literature has only peripherally touched upon its importance. Rather than emphasizing its significance, the resulting auto-oxidized molecule, L-cystine, has often been classified as “used-up cysteine” or classified exclusively as a biomarker of oxidative stress, and as an indication of a pathological oxidized state. See Dhawan et al. (above); Patel et al.'s article entitled “Oxidative stress is associated with impaired arterial elasticity.”

Research has recently demonstrated that L-cystine exemplifies a pleiotropic paradox, and its role is vital in the synthesis of glutathione and certain other concomitant but unexpected results, such as the activation of the vital gene Nrf2. See the aforementioned publications by Sinha et al. On closer examination and upon extensive university research, other dimensions to L-cystine have been verified. It has been documented in the literature and in university research that L-cystine is stable and neutral and water insoluble, as compared to L-cysteine, which is highly oxidizable and somewhat toxic to the body. See Janaky et al. (“Mechanisms of L-cysteine Neurotoxicity,” Neurochemical Research. Vol. 25. Nos. 9/10, 2000, pp 1397-1405); Dilger et al. “Excess dietary L-Cysteine, but not L-cystine, is lethal for chicks but not for rats or pigs,” Journal of Nutrition, 2007 Feb.; 137(2):331 - - - 8); Crum et al. Presentation before American Chemical Society, Aug. 21, 2007 entitled “sulfenic acid, sulfinic acid, sulfonic acid.”

Although L-cysteine is the crucial and most valuable functional detox moiety of glutathione (considered the body's master antioxidant), getting the L-cysteine into the intracellular space, where it could enter into the glutathione synthesis chain, was for a long time considered a scientific enigma. When a highly oxidizable molecule such as L-cysteine, which has toxic features, is also vital for the physiological synthesis of glutathione, it can be comprehended that nature has adapted an evolutionary advantage to auto-oxidation of that molecule (L-cysteine) for its safe carriage to the intracellular milieu where it can be utilized for the physiological synthesis of glutathione. There have been other methods tried to get the highly-oxidizable L-cysteine into the cytosol, but with limited results. The synthetic ester, N-acetyl cysteine, has been used by scientists to reduce the high reactivity and high oxidizability of the solo L-cysteine, so as to enable it to reach the intracellular glutathione synthesis chain with less reactivity and less oxidizability. Large protein molecules from non-denatured whey have also been used in an effort to keep the highly reactive, highly oxidizable but rate-limiting L-cysteine “in check” until it could enter the intracellular space of the glutathione synthesis milieu.

The inventor of the instant application utilized the advantage of L-cystine's disulfide bond as the safe physiological carrier of L-cysteine as the method to accomplish this vital L-cysteine delivery role. Upon arrival at the cell wall, substrate-specific enzymes, oxidoreductase, and thioltransferase at the cell membranes and in the cytosolic milieu decouple the tenacious disulfide bond of L-cystine. The decoupling of the disulfide bond permits the released, free form L-cysteine to be available for incorporation into the reducing cytosolic media of the intracellular environment. Also present in the intracellular space is the substrate specific gamma-glutamylcysteine synthetase, readily available to catalyze a unity or L-cysteine to L-glutamic acid. Given the wide-spread perception that the disulfide bond were essentially “fixed” or “irreversible,” scientists had not realized or formulated the diverse physiological and biochemical potential of L-cystine in glutathione synthesis. A typical comment or conclusion was that “Cystine is not suitable as an intracellular delivery agent (for L-cysteine) because of its marked insolubility.” (P. 317 Methods in Enzymology, Volume 143.) Misconceptions have been made in limiting the functions of L-cystine to only a measurement or biomarker for oxidative stress. Attempts have been made to force a parallel interpretation of intracellular glutathione to extracellular L-cystine, because they both contain the sulfhydryl radical and are active in various redox functions. The sulfhydryl group in free form L-cysteine functions with different properties when it is in a solo amino acid as compared to when its sulfhydryl group is a moiety of glutathione.

In summary, solo L-cysteine has different, complex and paradoxical functions for its sulfhydryl that distinguish it from the sulfhydryl functions when it is a moiety of glutathione. A recent study has interpreted results that need further clarification. See, Patel et al., (Oxidative Stress is associated with impaired arterial elasticity.” Atherosclerosis. 2011), which is incorporated by reference. Patel states “Non-free radical oxidative stress was assessed as plasma oxidized and reduced amino-thiol levels (cysteine/cysteine, glutathione/GSSG) and their ratios (redox, potentials), and free radical oxidative stress as derivatives of reactive oxygen metabolites (dROMs).”

In accordance with the foregoing analysis, the inventor herein have recognized that if physiologically synthesized by a step-by-step physiological synthesis pathway is followed, the resulting glutathione with only three amino acids and a cofactor (e.g., a selenium source such as selenomethionine or selenocysteine or a combination thereof in any ratio) can outmaneuver the other antioxidant systems. The human clinical trial for this adaptation has been received favorably. See, for example, National Clinical Trials with the accession No. NCT01251315 and references related thereto.

In accordance with the present invention, the physiologically active intracellular glutathione described hereinbefore, in order to be immunologically protective in the long term, is synthesized in a step-by-step process. This involves provision of the three amino acid components (either simultaneously or separately), which are then taken up by the respective transporters and synthesized intracellularly. The cysteine component of GSH is preferably provided in a reduced, dipeptide (cystine) form.

Embodiments of the instant invention indicate that the physiological glutathione synthesized by the aforementioned step-by-step process is better than whole glutathione molecule, e.g., with regard to chelation (and sequestration) of metal ions and the concomitant inhibition of viral replication. In contrast, whole glutathione is less effective because the provision thereof can throw the substrate specific enzymes into a vestigiality of disuse and further result in the dysregulation of the regulatory mechanism of physiological glutathione quantification levels. In fact, credible evidence suggests that the composition of the instant invention comprising the three component amino acids (glutamine or glutamate, cystine and glycine) and a cofactor (e.g., a selenium source such as selenomethionine or selenocysteine or a combination thereof in any ratio) is superior to other cellular thiol-antioxidants such as N-acetyl cysteine, α-lipoic acid, etc.

If the immune system is weakened and the glutathione is low or vestigially compromised, then the host's protective edge is impaired, unable to take molecular control of the zinc in the case of Ebola, and conceding the advantage to the pathogenic virus. If the immune system is robust with physiologically constituted glutathione, the glutathione will provide metallothioneine-like protection and the host triumphs against pathogenic viruses biochemically and physiologically. The sulfhydryl of physiologically constituted glutathione is more effective for the conjugation of zinc, than the pathogenic virus for the adherence of this vital metal.

### Reducing Metal Toxicity

In related embodiments, the instant invention provides novel and inventive means for reducing the toxicity caused by metal ions (e.g., due to dysregulation of iron, nickel and/or zinc homeostasis or due to pathogenic conditions) on biological systems. The methods involving contacting the afflicted biological system, which is a cell, a tissue, an organ, or an organism (e.g., a human or a non-human animal) with the aforementioned compositions. Preferably, the compositions comprise glycine, glutamate source (glutamine or glutamic acid) and L-cystine, optionally together with a selenium source (e.g., selenomethionine, selenocysteine, or selenium particles). Further optionally, the compositions may contain additional chelator of Zn²⁺, Fe²⁺ or Ni²⁺, or a combination of such chelators. Preferably, the chelators are bio-compatible and have dissociation constants that are lower than those of proteins which bind to the metal ions (e.g., RR or ZFP). Representative examples of such chelators include, for example, zinc chelators such as N,N,N′,N′-tetrakis(2-pyridylmethyl)-ethylenediamine (TPEN), DPESA, TPESA, ethylenediaminetetraacetic acid (EDTA), ethylene glycol-bis(2-aminoethylether)-N,N,N′,N′-tetraacetic acid (EGTA), 1,2-bis(o-aminophenoxy)ethane-N,N,N′,N′-tetraacetic acid (BAPTA), and ethylenediamine-N,N′-diacetic-N,N′-di-3-propionic (EDPA), etc. and iron chelators include diethylene triamine pentaacetic acid (DETAPAC), dipyridyl, pyridoxal isonicotinoyl hydrazone (PIH), desferrioxamine (DFO), deferiprone (DFP) or deferasirox (DFS). A combination of such chelators may also be employed.

### DEFINITIONS

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All technical and patent publications cited herein are incorporated herein by reference in their entirety. Nothing herein is to be construed as an admission that the invention is not entitled to antedate such disclosure by virtue of prior invention.

The practice of the present invention will employ, unless otherwise indicated, conventional techniques of tissue culture, immunology, molecular biology, microbiology, cell biology and recombinant DNA, which are within the skill of the art. See, e.g., Sambrook and Russell eds. (2001) Molecular Cloning: A Laboratory Manual, 3.sup.rd edition; the series Ausubel et al. eds. (2007) Current Protocols in Molecular Biology; the series Methods in Enzymology (Academic Press, Inc., N. Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5.sup.th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Herzenberg et al. eds (1996) Weir's Handbook of Experimental Immunology; Manipulating the Mouse Embryo: A Laboratory Manual, 3.sup.rd edition (Cold Spring Harbor Laboratory Press (2002)).

All numerical designations, e.g., pH, temperature, time, concentration, and molecular weight, including ranges, are approximations which are varied (+) or (−) by increments of 0.1 or 1.0, where appropriate. It is to be understood, although not always explicitly stated that all numerical designations are preceded by the term “about”. It also is to be understood, although not always explicitly stated, that the reagents described herein are merely exemplary and that equivalents of such are known in the art.

As used in the specification and claims, the singular forms “a”, “an” and “the” include plural references unless the context clearly dictates otherwise. For example, the term “a cell” includes a plurality of cells, including mixtures thereof.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of this invention or process steps to produce a composition or achieve an intended result. Embodiments defined by each of these transition terms are within the scope of this invention.

As is known to those of skill in the art, there are six classes of viruses. The DNA viruses constitute classes I and II. The RNA viruses and retroviruses make up the remaining classes. Class III viruses have a double-stranded RNA genome. Class IV viruses have a positive single-stranded RNA genome, the genome itself acting as mRNA Class V viruses have a negative single-stranded RNA genome used as a template for mRNA synthesis. Class VI viruses have a positive single-stranded RNA genome but with a DNA intermediate not only in replication but also in mRNA synthesis. Retroviruses carry their genetic information in the form of RNA; however, once the virus infects a cell, the RNA is reverse-transcribed into the DNA form which integrates into the genomic DNA of the infected cell. The integrated DNA form is called a provirus.

“Virus” includes any infectious agent that relies on a “host” for replication. Included in this definition are virions, viral particles, and mature viruses, which are either naturally-occurring or synthetic in nature. Representative examples include members of Arenaviridae, Reoviridae, Rotaviridae, Retroviridae, Papillomavirinae, Influenza, Adenoviridae, Flaviviridae (Hepatitis C), Herpesviridae, Filoviridae (e.g., Ebola virus and Marburg virus), Pneumovirinae (e.g., RSV), Orthomyxoviridae (Influenza viruses), etc. In this context, it should be recognized that Ebola virus is a member of the Filovirus family. Others include, but are not limited to Marburg viruses, Cucvavirus and the like.

The “infectivity” of a virus intends the ability of the virus to infect the host. Viral infection is affected by the infectivity, replicative fitness, and the ability of the virus to evade the host's immune response and develop resistance to antivirals.

“Chelation” intends the formation or presence of two or more separate bindings between a polydentate ligand and a single central atom. A “chelant” or “chelator” refers to a chemical that form a soluble and complex molecule with certain metal ions, inactivating the ions so that they cannot normally react with other elements or ions to produce precipitates or scale.

A “zinc chelator” refers to a chelator that chelates with zinc ions, e.g., Zn²⁺. An “iron chelator” refers to a chelator that chelates with iron ions, e.g., Fe²⁺/Fe³⁺. Non-limiting examples of zinc chelators include N,N,N′,N′-tetrakis(2-pyridylmethyl)-ethylenediamine (TPEN), DPESA, TPESA, ethylenediaminetetraacetic acid (EDTA), ethylene glycol-bis(2-aminoethylether)-N,N,N′,N′-tetraacetic acid (EGTA), 1,2-bis(o-aminophenoxy)ethane-N,N,N′,N′-tetraacetic acid (BAPTA), and ethylenediamine-N,N′-diacetic-N,N′-di-3-propionic (EDPA), etc. Non-limiting examples of iron chelators include diethylene triamine pentaacetic acid (DETAPAC), dipyridyl, pyridoxal isonicotinoyl hydrazone (PIH), desferrioxamine (DFO), deferiprone (DFP) or deferasirox (DFS) which chelates iron and inhibits metal-catalyzed reactions that produce free radical and non-radical reactive species.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. Unless otherwise specified or required, any embodiment of this invention that is a polynucleotide encompasses both the double-stranded form and each of two complementary single-stranded forms known or predicted to make up the double-stranded form.

A “polynucleotide” is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics.

A “gene” refers to a polynucleotide containing at least one open reading frame (ORF) that is capable of encoding a particular polypeptide or protein after being transcribed and translated. Any of the polynucleotide or polypeptide sequences described herein may be used to identify larger fragments or full-length coding sequences of the gene with which they are associated. Methods of isolating larger fragment sequences are known to those of skill in the art.

The term “express” refers to the production of a gene product. As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA.

A “gene product” or alternatively a “gene expression product” refers to the amino acid (e.g., peptide or polypeptide) generated when a gene is transcribed and translated.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof.

A “probe” when used in the context of polynucleotide manipulation refers to an oligonucleotide that is provided as a reagent to detect a target potentially present in a sample of interest by hybridizing with the target. Usually, a probe will comprise a detectable label or a means by which a label can be attached, either before or subsequent to the hybridization reaction. Alternatively, a “probe” can be a biological compound such as a polypeptide, antibody, or fragments thereof that is capable of binding to the target potentially present in a sample of interest. “Detectable labels” include, but are not limited to radioisotopes, fluorochromes, chemiluminescent compounds, dyes, and proteins (e.g., enzymes).

The term “propagate” means to grow a cell or population of cells. The term “growing” also refers to the proliferation of cells in the presence of supporting media, nutrients, growth factors, support cells, or any chemical or biological compound necessary for obtaining the desired number of cells

The term “culturing” refers to the in vitro propagation of cells or organisms on or in media of various kinds. It is understood that the descendants of a cell grown in culture may not be completely identical (i.e., morphologically, genetically, or phenotypically) to the parent cell.

A “composition” is intended to mean a combination of an active ingredient (e.g., individual components of the aforementioned metallothionine analogs) and another compound or composition, wherein the second component may be inert (e.g., a carrier) or active (e.g., another metal chelator).

For convenience, the term “selenium” is sometimes used hereinafter to include any of the various water-soluble selenium products which can be transported through the mucosal membrane in the practice of this invention. It will be understood, however, that the particular forms of selenium compounds set forth herein are not to be considered limitative. Other selenium compounds, which exhibit the desired activity and are compatible with the other components in the mixture and are non-toxic, can be used in the practice of the invention. Many of them are available commercially.

An “antioxidant” is a molecule capable of slowing or preventing the oxidation of other molecules. Oxidation is a chemical reaction that transfers electrons from a substance to an oxidizing agent. Oxidation reactions can produce free radicals, which cause oxidative stress and start chain reactions that damage cells. “Oxidative stress” is caused by an imbalance between the production of reactive oxygen and a biological system's ability to readily detoxify the reactive intermediates or easily repair the resulting damage. All forms of life maintain a reducing environment within their cells. This reducing environment is preserved by enzymes that maintain the reduced state through a constant input of metabolic energy. Disturbances in this normal redox state can cause toxic effects through the production of peroxides and free radicals that damage all components of the cell, including proteins, lipids, and DNA. Antioxidants terminate these chain reactions by removing free radical intermediates, and inhibit other oxidation reactions by being oxidized themselves. Examples of antioxidants include, but are not limited to, glutathione, N-acetylcysteine, ascorbic acid, vitamin E, beta-carotene, a polyphenol, flavonoid and an agent that decreases the generation of free radical and non-radical reactive species, including, for example, a CYP2E1 inhibitor, an NAD(P)H oxidase inhibitor or a nitric oxide synthase inhibitor.

“Ascorbic acid” or “vitamin C” refers a monosaccharide antioxidant found in both animals and plants. As one of the enzymes needed to make ascorbic acid has been lost by mutation during human evolution, it must be obtained from the diet and is a vitamin. Most other animals are able to produce this compound in their bodies and do not require it in their diets. In cells, it is maintained in its reduced form by reaction with glutathione, which can be catalyzed by protein disulfide isomerase and glutaredoxins. Ascorbic acid is a reducing agent and can reduce, and thereby neutralize, reactive oxygen species such as hydrogen peroxide. In addition to its direct antioxidant effects, ascorbic acid is also a substrate for the antioxidant enzyme ascorbate peroxidase, a function that is particularly important in stress resistance in plants. Ascorbic acid is present at high levels in all parts of plants and can reach concentrations of 20 millimolar in chloroplasts. Ascorbic acid can be used in combination with iron chelator because it can act as a pro-oxidant in the presence of iron by reducing iron to Fe2+, which would increase the generation of potent oxidants that would damage the nucleic acids.

“Glutathione” intends a cysteine-containing peptide found in most forms of aerobic life. It is not required in the diet and is instead synthesized in cells from its constituent amino acids. Glutathione has antioxidant properties since the thiol group in its cysteine moiety is a reducing agent and can be reversibly oxidized and reduced. In cells, glutathione is maintained in the reduced form by the enzyme glutathione reductase and in turn reduces other metabolites and enzyme systems, such as ascorbate in the glutathione-ascorbate cycle, glutathione peroxidases and glutaredoxins, as well as reacting directly with oxidants. In some organisms glutathione is replaced by other thiols, such as by mycothiol in the Actinomycetes, or by trypanothione in the kinetoplastids. Plasma and liver glutathione concentrations can be raised by oral administration of S-adenosylmethionine (SAMe). Glutathione precursors rich in cysteine include N-acetylcysteine (NAC) and undenatured whey protein, and these supplements have been shown to increase glutathione content within the cell. N-Acctylcysteine, is available both as a drug and as a generic supplement. Alpha Lipoic Acid has also been shown to restore intracellular glutathione. Melatonin has been shown to stimulate a related enzyme, glutathione peroxidase, and silymarin or milk thistle has also demonstrated an ability to replenish glutathione levels. Of all of these methods, the two methods that are the most thoroughly researched for efficacy in raising intracellular glutathione are variants of cysteine. N-acetyl-cysteine, which is a pharmaceutical over the counter drug, and bonded cysteine as is found in the undenatured whey protein nutraceutical, are both proven to be efficacious in raising glutathione values. Also, glutathione can be supplied in the form of glutathione esters.

“Melatonin”, known chemically as N-acetyl-5-methoxytryptamine, refers to a naturally occurring hormone found in animals and in some other living organisms, including algae.

“Vitamin E” is the collective name for a set of eight related tocopherols and tocotrienols, which are fat-soluble vitamins with antioxidant properties. A non-limiting example, .alpha.-tocopherol has been most studied as it has the highest bioavailability, with the body preferentially absorbing and metabolizing this form. .alpha.-tocopherol protects membranes from oxidation by reacting with lipid radicals produced in the lipid peroxidation chain reaction. This removes the free radical intermediates and prevents the propagation reaction from continuing. This reaction produces oxidized .alpha.-tocopheroxyl radicals that can be recycled back to the active reduced form through reduction by other antioxidants, such as ascorbate, retinol or ubiquinol. This is in line with findings showing that .alpha.-tocopherol, but not water-soluble antioxidants, efficiently protects glutathione peroxidase 4 (GPX4)-deficient cells from cell death. Vitamin E is available from dietary sources such as asparagus, avocado, egg, milk, nuts, seeds, spinach, unheated vegetable oil, wheat germ or wholegrain foods.

A “pharmaceutical composition” is intended to include the combination of an active polypeptide, polynucleotide or antibody with a carrier, inert or active such as a solid support, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, sec Martin (1975) Remington's Pharm. Sci., 15th Ed. (Mack Publ. Co., Easton).

A “subject,” “individual” or “patient” is used interchangeably herein, and refers to a vertebrate, preferably a mammal, more preferably a human. Mammals include, but are not limited to, murines, rats, rabbit, simians, bovines, ovine, porcine, canines, feline, farm animals, sport animals, pets, equine, and primate, particularly human. Besides being useful for human treatment, the present invention is also useful for veterinary treatment of companion mammals, exotic animals and domesticated animals, including mammals, rodents, and the like which is susceptible to viral infection. In one embodiment, the mammals include horses, dogs, and cats. In another embodiment of the present invention, the human is an adolescent or infant under the age of eighteen years of age.

The terms “disease,” “disorder,” and “condition” are used inclusively and refer to any condition mediated at least in part by infection by a pathogenic agent such as viruses, bacteria or the like.

As used herein, the term “treatment” is defined as the application or administration of a therapeutic agent to a patient, or application or administration of a therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease or the predisposition toward disease. “Treating” or “treatment” of a disease includes: (1) preventing the disease, i.e., causing the clinical symptoms of the disease not to develop in a patient that may be predisposed to the disease but does not yet experience or display symptoms of the disease; (2) inhibiting the disease, i.e., arresting or reducing the development of the disease or its clinical symptoms; or (3) relieving the disease, i.e., regression of the disease or its clinical symptoms.

The term “suffering” as it related to the term “treatment” refers to a patient or individual who has been diagnosed with or is predisposed to infection or a disease incident to infection. A patient may also be referred to being “at risk of suffering” from a disease because of active or latent infection. This patient has not yet developed characteristic disease pathology.

An “effective amount” is an amount sufficient to effect beneficial or desired results. An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents of the present invention for any particular subject depends upon a variety of factors including the activity of the specific compound employed, the age, body weight, general health, sex, and diet of the subject, the time of administration, the rate of excretion, the drug combination, and the severity of the particular disorder being treated and form of administration. Treatment dosages generally may be titrated to optimize safety and efficacy. Typically, dosage-effect relationships from in vitro and/or in vivo tests initially can provide useful guidance on the proper doses for patient administration. In general, one will desire to administer an amount of the compound that is effective to achieve a serum level commensurate with the concentrations found to be effective in vitro. Determination of these parameters is well within the skill of the art. These considerations, as well as effective formulations and administration procedures are well known in the art and are described in standard textbooks. Consistent with this definition, as used herein, the term “therapeutically effective amount” is an amount sufficient to inhibit RNA virus replication in vitro or in vivo. “Prophylactically effective” as used herein means the amount of the composition which is sufficient to achieve the desired result, for example, to reduce the incidence of viral infection in a particular subject or a subject population.

As used herein, the term “reduced” intends a lower level as compared to a control or a prior measurement or value. In one aspect, a reduced mutation rate of an RNA virus in a cell treated with an iron chelator or an antioxidant refers to a level of mutation rate that is lower than the level of mutation rate of the RNA virus in a cell not treated with the iron chelator or the antioxidant or alternatively, prior to such treatment. In another aspect, it is a lower mutation rate as compared to treatment with another, different agent, alone or in combination with the iron chelator or the antioxidant. Reduced intends a reduction by at least about 5%, or alternatively about 10%, or alternatively about 15%, or alternatively about 20%, or alternatively about 25%, or alternatively about 30%, or alternatively about 35%, or alternatively about 40%, or alternatively about 45%, or alternatively about 50%, or alternatively about 55%, or alternatively about 60%, or alternatively about 65%, or alternatively about 70%, or alternatively about 75%, or alternatively about 80%, or alternatively about 85%, or alternatively about 90%, or alternatively about 95%, or alternatively or about 100% as compared to a control or prior measurement or value.

As used herein, the term “enhanced” intends a higher level as compared to a control or a prior measurement or value. In one aspect, an enhanced efficacy of an agent or a therapy to reduce or prevent infection of a cell by an RNA virus, which cell is treated with an iron chelator or an antioxidant, is a higher efficacy as compared to the agent or therapy to reduce or prevent infection of the cell by the RNA virus, which cell is not treated with the iron chelator or the antioxidant. In another aspect, it is a higher efficacy as compared to treatment with another, different agent, alone or in combination with the iron chelator or the antioxidant. Enhanced intends an increase by at least about 5%, or alternatively about 10%, or alternatively about 15%, or alternatively about 20%, or alternatively about 25%, or alternatively about 30%, or alternatively about 35%, or alternatively about 40%, or alternatively about 45%, or alternatively about 50%, or alternatively about 55%, or alternatively about 60%, or alternatively about 65%, or alternatively about 70%, or alternatively about 75%, or alternatively about 80%, or alternatively about 85%, or alternatively about 90%, or alternatively about 95%, or alternatively or about 100%, as compared to a control or prior measurement or value.

“Pharmaceutically acceptable” means one that is generally recognized as safe, approved by a regulatory agency of the federal or a state government or listed in the U.S. Pharmacopeia or other generally recognized pharmacopeia for use in animals, and more particularly in humans.

The term “administration” shall include without limitation, administration by oral, parenteral (e.g., intramuscular, intraperitoneal, intravenous, ICV, intracisternal injection or infusion, subcutaneous injection, or implant), by inhalation spray nasal, vaginal, rectal, sublingual, urethral (e.g., urethral suppository) or topical routes of administration (e.g., gel, ointment, cream, aerosol, etc.) and can be formulated, alone or together, in suitable dosage unit formulations containing conventional non-toxic pharmaceutically acceptable carriers, adjuvants, excipients, and vehicles appropriate for each route of administration. The invention is not limited by the route of administration, the formulation or dosing schedule.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

### Specific Embodiments

A few of the many embodiments encompassed by the present description are summarized in the following numbered paragraphs. The numbered paragraphs are self-referential. In particular, the phase “in accordance with any of the foregoing or the following” used in these paragraphs refers to the other paragraphs. The phrase means in the following paragraphs embodiments herein disclosed include both the subject matter described in the individual paragraphs taken alone and the subject matter described by the paragraphs taken in combination. In this regard, the purpose in setting forth the following paragraphs to describe various aspects and embodiments particularly by the paragraphs taken in combination. That is, the paragraphs are a compact way of setting out and providing explicit written description of all the embodiments encompassed by them individually and in combination with one another. As such, any subject matter set out in any of the following paragraphs, alone or together with any other subject matter of any one or more other paragraphs, including any combination of any values therein set forth taken alone or in any combination with any other value set forth, may be presented.

### Formulations/Compositions

Composition 1. A composition comprising a glutathione (GSH) precursor and a selenium source.

Composition 2. The composition in accordance with the foregoing or the following, wherein the glutathione precursor comprises glycine, L-cystine and a glutamate source.

Composition 3. The composition in accordance with the foregoing or the following, wherein the glutathione precursor comprises glycine, L-cystine and glutamate.

Composition 4. The composition in accordance with the foregoing or the following, wherein the glutamine source is glutamate (Glu) or glutamine (Gln).

Composition 5. The composition in accordance with the foregoing or the following, which is a pharmaceutical composition comprising a carrier, a solvent, an excipient, a surfactant or an emollient and optionally further comprising an additional pharmaceutical agent.

Composition 6. The composition in accordance with the foregoing or the following, wherein the selenium source is selenonomethionine, selenite, methylselenocysteine, or selenium nanoparticles.

Composition 7. The composition in accordance with the foregoing or the following, further comprising an additional pharmaceutical agent which is N-acetylcysteine, vitamin C, vitamin E, α-lipoic acid, folic acid, vitamins B6 and B12, silibinin, resveratrol or a combination thereof.

Composition 8. The composition in accordance with the foregoing or the following, further comprising a metallothionine or a fragment thereof.

Composition 9. A combination comprising at least two of the aforementioned compositions.

Composition 10. A composition in accordance with the foregoing or the following, which is a pharmaceutical composition.

Composition 11. A composition in accordance with the foregoing or the following, further comprises a metal chelator.

Composition 12. A composition in accordance with the foregoing or the following, which further comprises a Zn²⁺ chelator, a Fe³⁺ chelator, a Ni²⁺ chelator, a combination thereof.

Composition 13. A composition in accordance with the foregoing or the following, wherein the chelator is N,N,N′,N′-tetrakis(2-pyridylmethyl)-ethylenediamine (TPEN), DPESA, TPESA, ethylenediaminetetraacetic acid (EDTA), ethylene glycol-bis(2-aminoethylether)-N,N,N′,N′-tetraacetic acid (EGTA), 1,2-bis(o-aminophenoxy)ethane-N,N,N′,N′-tetraacetic acid (BAPTA), and ethylenediamine-N,N′-diacetic-N,N′-di-β-propionic (EDPA), diethylene triamine pentaacetic acid (DETAPAC), dipyridyl, pyridoxal isonicotinoyl hydrazone (PIH), desferrioxamine (DFO), deferiprone (DFP) or deferasirox (DFS) or a combination thereof.

Composition 14. A composition in accordance with the foregoing or the following, which further comprises an antiviral selected from the group consisting of abacavir, aciclovir, acyclovir, adefovir, amantadine, amprenavir, arbidol, atazanavir, atripla, brivudine, cidofovir, combivir, darunavir, delavirdine, didanosine, docosanol, edoxudine, efavirenz, emtricitabine, enfuvirtide, entecavir, entry inhibitors, famciclovir, fixed dose combinations, fomivirsen, fosamprenavir, foscarnet, fosfonet, fusion inhibitors, ganciclovir, gardasil, ibacitabine, imunovir, idoxuridine, imiquimod, indinavir, inosine, integrase inhibitors, interferon type III, interferon type II, interferon type I, interferon, lamivudine, lopinavir, loviride, MK-0518, maraviroc, moroxydine, nelfinavir, nevirapine, nexavir, nucleoside analogues, oseltamivir, penciclovir, perarnivir, pleconaril, podophyllotoxin, protease inhibitors, reverse transcriptase inhibitors, ribavirin, rimantadine, ritonavir, saquinavir, Stavudine, synergistic enhancers, tenofovir, tenofovir disoproxil, tipranavir, trifluridine, trizivir, tromantadine, truvada, valaciclovir, valganciclovir, vicriviroc, vidarabine, viramidine, zalcitabine, zanamivir, and zidovudine.

### Kits

Kit 1. A kit comprising, in one or separate compartments or packages, a glutathione (GSH) precursor and a selenium source, optionally together with an excipient, carrier or oil.

Kit 2. The kit in accordance with any of the foregoing or the following, comprising the glutathione precursor in one compartment and a selenium source in another compartment.

Kit 3. The kit in accordance with any of the foregoing or the following, comprising an additional pharmaceutical agent which is N-acetylcysteine, vitamin C, vitamin E, α-lipoic acid, folic acid, vitamins B6 and B12, silibinin, resveratrol or a combination at least two of the additional agents.

Kit 4. The kit in accordance with any of the foregoing or the following, further comprising instructions for formulating a composition comprising said glutathione (GSH) precursor and a selenium source.

Kit 5. The kit in accordance with any of the foregoing or the following, further comprising instructions for using the components, either individually or together, for the treatment of pathogenic diseases.

Kit 6. The kit in accordance with any of the foregoing or the following, further comprising instructions for using the components, either individually or together, for the treatment of viral diseases.

Kit 7. The kit in accordance with any of the foregoing or the following, further comprising instructions for using the components, either individually or together, for reducing the incidence of viral diseases.

Kit 8. The kit in accordance with any of the foregoing or the following, further comprising a metallothioneine or a fragment thereof.

Kit 9. The kit in accordance with any of the foregoing or following, further comprising an antiviral agent selected from the group consisting of abacavir, aciclovir, acyclovir, adefovir, amantadine, amprenavir, arbidol, atazanavir, atripla, brivudine, cidofovir, combivir, darunavir, delavirdine, didanosine, docosanol, edoxudine, efavirenz, emtricitabine, enfuvirtide, entecavir, entry inhibitors, famciclovir, fixed dose combinations, fomivirsen, fosamprenavir, foscarnet, fosfonet, fusion inhibitors, ganciclovir, gardasil, ibacitabine, imunovir, idoxuridine, imiquimod, indinavir, inosine, integrase inhibitors, interferon type III, interferon type II, interferon type I, interferon, lamivudine, lopinavir, loviride, MK-0518, maraviroc, moroxydine, nelfinavir, nevirapine, nexavir, nucleoside analogues, oseltamivir, penciclovir, peramivir, pleconaril, podophyllotoxin, protease inhibitors, reverse transcriptase inhibitors, ribavirin, rimantadine, ritonavir, saquinavir, stavudine, synergistic enhancers, tenofovir, tenofovir disoproxil, tipranavir, trifluridine, trizivir, tromantadine, truvada, valaciclovir, valganciclovir, vicriviroc, vidarabine, viramidine, zalcitabine, zanamivir, and zidovudine.

### Method of Treating

Treatment 1. A method of treating a disease associated with viral infection or reducing the incidence of infection associated with viral infection in a subject in need thereof comprising employing the composition in accordance with any of the foregoing or the following.

Treatment 2. The method for the treatment or reducing the incidence of a viral disease in a subject in accordance with any of the foregoing or the following, which is for the treatment of a viral disease.

Treatment 3. The method for the treatment or reducing the incidence of a viral disease in a subject in accordance with any of the foregoing or the following, wherein said composition additionally comprises a pharmaceutically acceptable carrier, excipient, emollient, surfactant or solvent.

Treatment 4. The method for the treatment or reducing the incidence of a viral disease in a subject in accordance with any of the foregoing or the following, wherein said composition is a pharmaceutical composition for oral administration, topical administration, nasal administration, sublingual administration, buccal administration, intravenous administration, surgical administration, anal administration or vaginal administration.

Treatment 5. The method for the treatment or reducing the incidence of a viral disease in a subject in accordance with any of the foregoing or the following, wherein said subject is a human or a non-human mammal.

Treatment 6. A method for the treatment or reducing the incidence of a viral disease in a subject in accordance with the foregoing or following, further comprising administering N-acetylcysteine, vitamin C, vitamin E, α-lipoic acid, folic acid, vitamins B6 and B12, silibinin, resveratrol or a combination thereof.

Treatment 7. A method for the treatment or reducing the incidence of a viral disease in a subject in need thereof, comprising administering to said subject a composition comprising a glutathione precursor and a selenium source.

Treatment 8. The method for the treatment or reducing the incidence of a viral disease in accordance with the foregoing or the following, wherein the glutathione precursor comprises glycine, L-cystine and a glutamate source.

Treatment 9. The method for the treatment or reducing the incidence of a viral disease in accordance with the foregoing or the following, wherein the selenium source is selenocysteine or selenomethionine.

Treatment 10. The method for the treatment or reducing the incidence of a viral disease in accordance with the foregoing or the following, wherein the composition further comprises a metallothioneine or a fragment thereof.

Treatment 11. The method for the treatment or reducing the incidence of a viral disease in accordance with the foregoing or the following, wherein the virus is of the family Arenaviridae, Reoviridae, Rotaviridae, Retroviridae, Papillomavirinae, Influenza, Adenoviridae, Flaviviridae (Hepatitis C), Herpesviridae, Filoviridae, Pneumovirinae, or Orthomyxoviridae.

Treatment 12. The method for the treatment or reducing the incidence of a viral disease in accordance with the foregoing or the following, wherein the virus is Ebola virus, Marburg virus, influenza virus, or respiratory syncytial virus (RSV).

Treatment 13. The method for the treatment or reducing the incidence of a viral disease in accordance with the foregoing or the following, further comprising administering, to a subject in need thereof, a metal chelator.

Treatment 14. The method for the treatment or reducing the incidence of a viral disease in accordance with the foregoing or the following, further comprising administering, to a subject in need thereof, an Fe³⁺ chelator, a Zn²⁺ chelator, an Ni²⁺ chelator, or a combination thereof.

Treatment 15. The method for the treatment or reducing the incidence of a viral disease in accordance with the foregoing or the following, wherein the chelator is N,N,N′,N′-tetrakis(2-pyridylmethyl)-ethylenediamine (TPEN), DPESA, TPESA, ethylenediaminetetraacetic acid (EDTA), ethylene glycol-bis(2-aminoethylether)-N,N,N′,N′-tetraacetic acid (EGTA), 1,2-bis(o-aminophenoxy)ethane-N,N,N′,N′-tetraacetic acid (BAPTA), and ethylenediamine-N,N′-diacetic-N,N′-di-3-propionic (EDPA), diethylene triamine pentaacetic acid (DETAPAC), dipyridyl, pyridoxal isonicotinoyl hydrazone (PIH), desferrioxamine (DFO), deferiprone (DFP) or deferasirox (DFS) or a combination thereof.

Treatment 16. The method for the treatment or reducing the incidence of a viral disease in accordance with the foregoing or the following, further comprising an antiviral agent selected from the group consisting of abacavir, aciclovir, acyclovir, adefovir, amantadine, amprenavir, arbidol, atazanavir, atripla, brivudine, cidofovir, combivir, darunavir, delavirdine, didanosine, docosanol, edoxudine, efavirenz, emtricitabine, enfuvirtide, entecavir, entry inhibitors, famciclovir, fixed dose combinations, fomivirsen, fosamprenavir, foscarnet, fosfonet, fusion inhibitors, ganciclovir, gardasil, ibacitabine, imunovir, idoxuridine, imiquimod, indinavir, inosine, integrase inhibitors, interferon type III, interferon type II, interferon type I, interferon, lamivudine, lopinavir, loviride, MK-0518, maraviroc, moroxydine, nelfinavir, nevirapine, nexavir, nucleoside analogues, oseltamivir, penciclovir, peramivir, pleconaril, podophyllotoxin, protease inhibitors, reverse transcriptase inhibitors, ribavirin, rimantadine, ritonavir, saquinavir, stavudine, synergistic enhancers, tenofovir, tenofovir disoproxil, tipranavir, trifluridine, trizivir, tromantadine, truvada, valaciclovir, valganciclovir, vicriviroc, vidarabine, viramidine, zalcitabine, zanamivir, and Zidovudine.

Treatment 17. The method for the treatment or reducing the incidence of a viral disease in accordance with the foregoing or the following, wherein the viral disease is cancer.

Treatment 18. The method for the treatment or reducing the incidence of a viral disease in accordance with the foregoing or the following, wherein the cancer is Kaposi's sarcoma, Burkett's lymphoma, adult T-cell leukemia, Merkel cell carcinoma, papilloma-virus induced cancers of cervix, vulva, vagina, penis, anus, and nasopharyngeal carcinoma.

### Methods of Reducing Toxicity

Toxicity 1. A method for reducing iron, nickel or zinc toxicity in a biological system, comprising contacting said biological system with the composition in accordance with the foregoing or following.

Toxicity 2. A method for reducing iron, nickel or zinc toxicity in a biological system, comprising administering to said subject a composition comprising a glutathione precursor and a selenium source.

Toxicity 3. The method for reducing iron, nickel or zinc toxicity in a biological system in accordance with the foregoing or the following, wherein the glutathione precursor comprises glycine, L-cystine and a glutamate source.

Toxicity 4. The method for reducing iron, nickel or zinc toxicity in a biological system in accordance with the foregoing or the following, wherein the selenium source is selenocysteine or selenomethionine.

Toxicity 5. The method for reducing iron, nickel or zinc toxicity in a biological system in accordance with the foregoing or the following, wherein the composition further comprises a metallothioneine or a fragment thereof.

Toxicity 6. The method for reducing iron, nickel or zinc toxicity in a biological system in accordance with the foregoing or the following, wherein the system is a cellular system, a tissue system, an organ system, or an organism.

Toxicity 7. The method for reducing iron, nickel or zinc toxicity in a biological system in accordance with the foregoing or the following, wherein the toxicity is due to dysregulated iron, nickel or zinc homeostasis.

Toxicity 8. The method for reducing iron, nickel or zinc toxicity in a biological system in accordance with the foregoing or the following, further comprising administering, to a subject in need thereof, a metal chelator.

Toxicity 9. The method for reducing iron, nickel or zinc toxicity in a biological system in accordance with the foregoing or the following, further comprising administering, to a subject in need thereof, an Fe³⁺ chelator, a Zn²⁺ chelator, an Ni²⁺ chelator, or a combination thereof.

Toxicity 10. The method for reducing iron, nickel or zinc toxicity in a biological system in accordance with the foregoing or the following, wherein the chelator is N,N,N′,N′-tetrakis(2-pyridylmethyl)-ethylenediamine (TPEN), DPESA, TPESA, ethylenediaminetetraacetic acid (EDTA), ethylene glycol-bis(2-aminoethylether)-N,N,N′,N′-tetraacetic acid (EGTA), 1,2-bis(o-aminophenoxy)ethane-N,N,N′,N′-tetraacetic acid (BAPTA), and ethylenediamine-N,N′-diacetic-N,N′-di-3-propionic (EDPA), diethylene triamine pentaacetic acid (DETAPAC), dipyridyl, pyridoxal isonicotinoyl hydrazone (PIH), desferrioxamine (DFO), deferiprone (DFP) or deferasirox (DFS) or a combination thereof.

Toxicity 10. A method for reducing iron, nickel or zinc toxicity in a biological system in accordance with the foregoing or following, further comprising administering N-acetylcysteine, vitamin C, vitamin E, α-lipoic acid, folic acid, vitamins B6 and B12, silibinin, resveratrol or a combination thereof.

According to one embodiment of the present invention there is provided a method of treatment of viral diseases comprising administering to a subject in need of such treatment an effective amount of a composition comprising components for increasing intracellular glutathione (GSH [reduced form] or GSSG [oxidized form]) and a selenium source. The individual components of the composition are disclosed in detail in Crum et al. (US patent app. pub. No. 2012-0029082), which is incorporated by reference herein in its entirety.

As detailed in the aforementioned Crum et al., the individual components of the compositions include: (1) three amino acids which serve as precursors of glutathione, i.e., glycine, L-cysteine (as L-cystine) and glutamate (which can, in turn, be provided in the form of glutamic acid or glutamine). These components are the precursors of the metallothionein analogs described herein.

The glutathione precursor includes, individual components, e.g., glutamic acid, cystine (as the cysteine source) and glycine, or one or more biological precursors thereof (e.g., glutamate [Glu] or glutamine [Gln] as a precursor of glutamic acid; cysteine [Cys], including modified cysteine derivatives such as N-acteylcysteine [NAC], as a source of cysteine for the cystine, etc.). Other usable forms of the GSH component compounds include, for example, salts, esters, anhydrides, tautomers or analogs of glutamic acid, cystine and glycine. The aforementioned components of the compositions of the instant invention can be administered simultaneously, sequentially or separately to a subject in need of such treatment.

All amino acids employed in this invention, except glycine which does not form optical isomers, are in the natural or L-form. The individual components of the metallothioneine analogs may be provided in singularity (e.g., as a mixture of the individual components in the desired ratio) or in one or more separate packages.

### Selenium Source

The compositions of the invention also include a selenium source, which serves as a co-factor in the synthesis of GSH. Selenium is one of numerous trace metals found in many foods. The compositions may optionally comprise a selenium containing amino acid such as selenomethionine or selenocysteine. The composition may also contain other amino acids, such as, for example, methionine, arginine, oxoproline, and the like. These optional components may be provided together with, or separate from, the individual components of GSH, i.e., glycine, cystine, and glutamate.

In the compositions of this invention, selenium may be employed as one of several non-toxic, water-soluble organic or inorganic selenium compounds capable of being absorbed through the mucosal membrane. Representative examples of the selenium source include, but are not limited to selenomethionine, selenite, methylselenocysteine, selenium nanoparticles, including salts, esters, anhydrides, tautomers or analogs, etc. of the individual selenium sources.

Representative examples of inorganic selenium compounds are aliphatic selenium metal salts containing selenium in the form of selenite or selenate anions. However, organic selenium compounds are also employable because they are normally less toxic than their inorganic counterparts. Other selenium compounds which may be mentioned by way of example include selenium cystine, selenium methionine, mono- and di-seleno carboxylic acids with about seven to eleven carbon atoms in the chain. Seleno-amino acid chelates are also useful. These selenium compounds may be considered for use in the present invention as selenium particles or salts thereof. Representative examples are known in the art. See Kojouri et al. “The Effects of Oral Consumption of Selenium Nanoparticles on Chemotactic and Respiratory Burst Activities of Neutrophils in Comparison with Sodium Selenite in Sheep,” Biol Trace Elem Res. May 2012; 146(2): 160-166.

Although any ratiometric amounts of the individual components of the GSH precursor may be employed, it will be apparent to those skilled in the art that the optimum ratio of glutamic acid to cystine to glycine in the novel compositions described herein is between 0.5:1.0:0.5 (or 1:2:1) to 1:0.5:1 (or 2:1:2), including all ratiometric values in between, e.g., 1.1:2.0:1.1, 1.2:2.0:1.2, 1.3:2.0:1.3, 1.4:2.0:1.4, 1.5:2.0:1.5, 1.6:2.0:1.6, 1.7:2.0:1.7, 1.8:2.0:1.8, 1.9:2.0:1.9, 1.0:1.0:1.0, 1.1:1.0:1.1, 1.2:1.0:1.2, 1.3:1.0:1.3, 1.4:1.0:1.4, 1.5:1.0:1.5, 1.6:1.0:1.6, 1.7:1.0:1.7, 1.8:1.0:1.8, 1.9:1.0:1.9, 2.0:1.0:2.0, etc. If an excess of any acid is used, it will presumably be of nutritional value or may simply be metabolized.

As will be apparent to the skilled artisan, owing to the toxicity of the selenium compound, the dosage units for mammalian administration by any selected route will cater to avoiding treatment either with single or multiple dosages of the toxic compound and the dosage of the selenium compound will be adjusted so that the total delivery does not reach the toxic limit of 400 μg/day for humans (Institute of Medicine, Food and Nutrition Board. Dietary Reference Intakes: Vitamin C, Vitamin E, Selenium, and Carotenoids. National Academy Press, Washington, D.C., 2000).

The recommended daily allowances for selenium as reported in The Pharmacological Basis of Therapeutics, 9^{th} Ed., The McGraw-Hill Companies, 1996 are shown in Table 1 below:

TABLE 1
Recommended daily allowances for selenium.

| Subject | Age/Years | Dose/μg |
|---|---|---|
| Infants | 0.0-0.5 | 10 |
| | 0.5-1.0 | 15 |
| Children | 1.0-3.0 | 20 |
| | 4.0-6.0 | 20 |
| | 7.0-10.0 | 30 |
| Males | 11.0-14.0 | 40 |
| | 15.0-18.0 | 50 |
| | 19.0-24.0 | 70 |
| | 25.0-50.0 | 70 |
| | 51+ | 70 |
| Females | 11.0-14.0 | 45 |
| | 15.0-18.0 | 50 |
| | 19.0-24.0 | 55 |
| | 25.0-50.0 | 55 |
| | 51+ | 55 |
| Pregnant | — | 65 |
| Lactating | 1st six mo. | 75 |
| | 2nd six mo. | 75 |

The recommended daily dosage for humans therefore ranges from 10 to 75 μg per day and any range or value in between, including, but not limited to, 15 to 70 μg/day, 20 to 60 μg/day, 25 to 50 μg/day, 30 to 40 μg/day, etc. For animals the range may be generally higher but will, of course, depend upon the animal and its size.

The precise amount of the therapeutically useful compositions of this invention for daily delivery and the duration of the period of such delivery will depend upon the professional judgment of the physician or veterinarian in attendance. Numerous factors will be involved in that judgment such as age, body weight, physical condition of the patient or animal and the ailment or disorder being treated.

It is important for the practice of this invention that the selenium as employed in the composition be capable of transport through the mucosal membrane of the patient under treatment. For this reason, water insoluble selenium compounds are not generally useful.

Preferably, the selenium is provided with L-methionine (e.g., selenomethionine) or with L-cystine (e.g., selenocystine). The provision of selenium as the latter allows accomplishment of two vital goals simultaneously, (a) provision of the selenium co-factor; and (b) provision of an additional safe source of L-cysteine.

In fact, the amount of selenium precursor employed in the novel compositions is only enough to provide a catalytic quantity of the element to activate the glutathione system. The catalytic quantity of selenium precursor utilized in the compositions of this invention is such that it will produce either in one dosage unit or in multiple dosage units sufficient elemental selenium to promote the production and activation of glutathione. Typically, this will be at or near the recommended daily allowance of selenium for the individual mammal under treatment. This amount will be well below the toxicity limit for elemental selenium. By way of non-limiting examples, a representative range of catalytic quantity of selenium is presented in the aforementioned Table 1, as shown to be effective based on the subject's age.

### Compositions

This invention provides pharmaceutical compositions used in the method of the invention. Such compositions comprise a therapeutically effective amount of combined glutamic acid (in the form of glutamate or glutamine), cystine (as the L-Cysteine source), glycine and a selenium precursor in a pharmaceutically acceptable carrier. The individual components may also be provided individually with a common carrier or different carriers.

The compositions which may be provided in bulk or dosage unit form are prepared in accordance with standard pharmaceutical practice and may contain excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Sterile liquids, such as water and oils, including those of petroleum, animal, vegetable or synthetic origin, such as peanut oil, soybean oil, mineral oil, and sesame oil may also be useful. The composition, if desired, can also contain minor amounts of wetting or emulsifying agents, coloring agents or buffering agents.

Buffering agents are sometimes used in the compositions of the invention to maintain a relatively constant hydrogen ion concentration in the mouth (pH about 7.5) or other point of entry. An appropriate buffering agent may be selected from numerous known reagents including, for example phosphate, carbonate and bicarbonate systems. Alpha-lactalbumin is useful because of its buffering properties. Additionally, it is non-toxic, water-soluble and contains appreciable amounts of the required amino acids.

The compositions may also contain mucous membrane penetration enhancers such as sodium lauryl sulphate, sodium dodecyl sulphate, cationic surfactants such as palmitoyl DL carnitine chloride, cetylpyridinium chloride, non-ionic surfactants such as polysorbale 80, polyoxyethylene 9-lauryl either, glyceryl monolaurate, polyoxyalkylenes, polyoxyethylene 20 cetyl ether, lipids such as oleic acid, bile salts such as sodium glycocholate, sodium taurocholate and related compounds.

Examples of these suitable carriers are described in Remington's Pharmaceutical Sciences, Nineteenth Edition (1990), Mack Publishing Company, Easton, Pa. in Handbook of Pharmaceutical Excipients, published by The American Pharmaceutical Association and The Pharmaceutical Society of Great Britain (1986) and the Handbook of Water-Soluble Gums and Resins, Ed. By R. L. Davidson, McGraw-Hill Book Co., New York, N.Y. (1980). Compositions and methods of manufacturing compositions capable of absorption through the mucosal tissues are taught in U.S. Pat. No. 5,288,497. These publications are incorporated by reference herein in their entirety. They can be readily employed by the skilled artisan to devise methods of delivery other than those specifically described in this disclosure.

### Dosages

For compounds, exemplary doses include milligram or microgram amounts of the compound per kilogram of subject or sample weight, for example, about 1 microgram per kilogram to about 500 milligrams per kilogram, about 100 micrograms per kilogram to about 5 milligrams per kilogram, or about 1 microgram per kilogram to about 50 micrograms per kilogram. It is understood that appropriate doses of a small molecule depend upon the potency of the small molecule with respect to the expression or activity to be modulated, particularly when one delivers the molecule directly to the cell cytosol. When one or more of these small molecules is to be administered to an animal (e.g., a human) in order to modulate expression or activity of a polypeptide or nucleic acid described herein, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, nasal, optical, and rectal administration. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Oral compositions optionally may include an inert diluent or an edible carrier. For the purpose of oral therapeutic administration, the active compound need not be but can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, PRIMOGEL, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Pharmaceutical compositions that are suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, CRMPHOR EL (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, other fluids configured to preserve the integrity of the viral capsid, and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, isotonic agents, for example, sugars, polyalcohols such as mannitol, sorbitol, and sodium chloride sometimes are included in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above.

The pharmaceutical compositions of the invention are most conveniently utilized in dosage units for oral administration. They may be used alone but are preferably provided as tablets, suitably sublingual tablets. Such tablets may be prepared in one a day form or for intermittent use throughout the day, for example every three hours.

For example, tablets will typically weigh from about 0.5 to 5.0 grams, including all ranges and values in between, for example, about 0.6 to 4.5 grams, about 0.7 to 4.0 grams, about 0.8 to 3.5 grams, 0.9 to 3.0 grams, 1.0 to 2.5 grams, 1.5 to 2.0 grams. Microtablets that are less than 0.5 grams are also contemplated by the instant invention. The tablets will contain a therapeutically effective amount of the essential ingredients together with the selected vehicle.

A particular advantage of the compositions of the invention is that they can be provided in a number of different forms and at dosage levels appropriate to the individual mammal being treated. For example, tablets, elixers, solutions, emulsions, powders, capsules and other forms can be provided for one a day treatment or successive treatments on the same day for animals or humans whether male or female, whether infant, adolescent or adult. The defining feature of this advantage is the amount of selenium precursor utilized since the other components are essentially non-toxic.

Referring to the table above, tablets and other forms of the immunoenhancing compositions can be prepared to provide any quantity of elemental selenium from less than 1.0 μg (e.g., 0.9 μg, 0.8 μg, 0.7 μg, 0.6 μg, 0.5 μg, 0.4 μg, 0.2 μg, 0.1 μg, 0.05 μg, 0.01 μg or less) to 7.5 μg or more (e.g., 8.0 μg, 9.0 μg, 10.0 μg, 15 μg, 20.0 μg, 40.0 μg, 100.0 μg, or more) including all values in between, for example, between 1.5 μg to 20 μg, between 2.0 μg to 15 μg, between 2.5 μg to 10 μg, between 1.5 μg to 7.5 μg, between 2.0 μg to 5.0 μg, etc. Herein it is understood that a tablet containing 10 μg of selenium methionine is capable of delivering 4 μg of elemental selenium, and 7.5 μg of selenium methionine is capable of delivering 3 μg of selenium. Tablets may be given several times per day to achieve the desired immune enhancing effect.

A one a day tablet weighing two grams may contain 200 mg or more (e.g., up to 200 mg, up to 300 mg, up to 500 mg, up to 1000 mg, up to 2000 mg, or more) of the composition (containing, for example, 5% to 10% by weight of the active ingredient). A similar tablet intended to be used every four hours may contain 50 mg to 100 mg or more of the therapeutically effective composition. Equivalent amounts of carrier and active components will be utilized in other compositions designed for other methods of administration.

### Formulations

The aforementioned compositions and combinations may be formulated to include suitable additives and further pharmaceutical ingredients. Examples of such additives include, but are not limited to, for example, coenzyme Q10 (CoQ10), ubiquinone, 7-keto dehydroepiandrosterone (7-keto DHEA), N-acetyl-cysteine, magnesium orotate or a combination thereof. See Hastings et al. (U.S. Pat. No. 6,368,617) and Richardson et al. (U.S. Pat. No. 6,207,190), which are incorporated by reference in parts pertinent thereto.

The compositions may include antiviral agents known in the art. Suitable antiviral agents include, for example, abacavir, aciclovir, acyclovir, adefovir, amantadine, amprenavir, arbidol, atazanavir, atripla, brivudine, cidofovir, combivir, darunavir, delavirdine, didanosine, docosanol, edoxudine, efavirenz, emtricitabine, enfuvirtide, entecavir, entry inhibitors, famciclovir, fixed dose combinations, fomivirsen, fosamprenavir, foscarnet, fosfonet, fusion inhibitors, ganciclovir, gardasil, ibacitabine, imunovir, idoxuridine, imiquimod, indinavir, inosine, integrase inhibitors, interferon type III, interferon type II, interferon type I, interferon, lamivudine, lopinavir, loviride, MK-0518, maraviroc, moroxydine, nelfinavir, nevirapine, nexavir, nucleoside analogues, oseltamivir, penciclovir, peramivir, pleconaril, podophyllotoxin, protease inhibitors, reverse transcriptase inhibitors, ribavirin, rimantadine, ritonavir, saquinavir, stavudine, synergistic enhancers, tenofovir, tenofovir disoproxil, tipranavir, trifluridine, trizivir, tromantadine, truvada, valaciclovir, valganciclovir, vicriviroc, vidarabine, viramidine, zalcitabine, zanamivir, and zidovudine. Exemplary antiviral agents are listed in, for example, U.S. Pat. Nos. 6,093,550 and 6,894,033; and also those listed in Table 2 of Sharma et al. (US patent app. Pub. No. 2010-0081713), the disclosures in which are incorporated by reference herein. Any combination of antiviral agents may also be used.

Certain biologics can be used for modifying a given biological response, the drug moiety delivered via the viral capsid is not to be construed as limited to classical chemical therapeutic agents. For example, the drug moiety may be a protein or polypeptide possessing a desired biological activity. Such proteins may include, for example, a toxin such as abrin, ricin A, pseudomonas cxotoxin, or diphtheria toxin; a polypeptide such as tumor necrosis factor, α-interferon, β-interferon, nerve growth factor, platelet derived growth factor, tissue plasminogen activator; or, biological response modifiers such as, for example, lymphokines, interleukin-1 (“IL-”), interleukin-2 (“IL-2”), interleukin-6 (“IL-6”), granulocyte macrophage colony stimulating factor (“GM-CSF”), granulocyte colony stimulating factor (“G-CSF”), or other growth factors. Alternatively, an antibody can be conjugated to a second antibody to form an antibody heteroconjugate.

Nucleic acid molecules can be inserted into viral capsids and used in gene therapy methods for treatment, including without limitation, cancer. Gene therapy capsids can be delivered to a subject by, for example, intravenous injection and local administration. Pharmaceutical preparations of gene therapy capsids can include a gene therapy capsid in an acceptable diluent, or can comprise a slow release matrix in which the gene delivery vehicle is imbedded.

### Delivery Agents

As indicated above, the presently preferred method of delivery for the compositions is oral, topical, sublingual or buccal. It is convenient to provide dosage units for such delivery in the form of pills, powders, lozenges or tablets such as gelled tablets, which will slowly dissolve in the mouth. Furthermore, for topical delivery, the formulation may be in the form that would be appropriate to the skin, such as lotions, unguents, emollients, creams, etc.

Sprays or drops will typically accomplish nasal delivery of the agents of the instant invention. Suppositories will be useful for rectal or vaginal delivery.

For administration by inhalation, the compounds are delivered in the form of an aerosol spray from pressured container or dispenser that contains a suitable propellant, e.g., a gas such as carbon dioxide, or a nebulizer.

Systemic administration can also be by transmucosal or transdermal means, including nasal and optical. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art. Delivery vehicles can also be prepared in the form of suppositories (e.g., with conventional suppository bases such as cocoa butter and other glycerides) or retention enemas for rectal delivery.

The present composition may include flavorings. Flavors may be based on peppermint oil, parsley, clove oil or a combination of the flavors.

### Dosimetry

In some embodiments oral or parenteral compositions are formulated in a dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Molecules which exhibit high therapeutic indices often are utilized. While molecules that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such molecules often lies within a range of circulating concentrations that include the ED50 with little or no toxicity. The dosage may vary within this range depending upon the dosage form employed and the route of administration utilized. For any molecules used in the methods described herein, the therapeutically effective dose can be estimated initially from cell culture assays. A dose may be formulated in animal models to achieve a circulating plasma concentration range that includes the IC.sub.50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography. Another example of effective dose determination for an individual is the ability to directly assay levels of “free” and “bound” compound in the serum of the test subject. Such assays may utilize antibody mimics and/or biosensors.

### Kits and Packs

Pharmaceutical compositions can be included in a kit, container, pack, or dispenser together with instructions for administration. Pharmaceutical compositions of active ingredients can be administered by any of the paths described herein for therapeutic and prophylactic methods for treatment. With regard to both prophylactic and therapeutic methods of treatment, such treatments may be specifically tailored or modified, based on knowledge obtained from pharmacogenomic analyses described herein. A therapeutic agent includes, but is not limited to, small molecules, peptides, antibodies, ribozymes, oligonucleotides, and analgesics.

Every document cited herein, including any cross-referenced or related patent or application is hereby incorporated herein by reference in its entirety unless expressly excluded or otherwise limited. The citation of any document is not an admission that it is prior art with respect to any invention disclosed or claimed herein or that it alone, or in any combination with any other reference(s), teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated, it would be well within the skill and expertise of those skilled in the art that various other changes and modifications can be made without departing from the spirit and scope of the invention. It is therefore intended to cover in the appended claims all such changes and modifications that are within the scope of this invention.

What is claimed is:

1. A method for the treatment or reducing the incidence of a viral disease in a subject in need thereof, comprising administering to said subject, a composition comprising a glutathione precursor and a selenium compound optionally together with a metallothionein or a fragment thereof.

2. The method of claim 1, wherein the glutathione precursor comprises glycine, L-cystine, and a glutamate source.

3. The method of claim 2, wherein the glutathione precursor comprises glycine, L-cystine, and glutamine.

4. The method of claim 2, wherein the glutamate source is glutamine or glutamic acid.

5. The method of claim 1, wherein the selenium compound is selenomethionine, selenite, methylselenocysteine or selenium nanoparticles.

6. The method of claim 1, further comprising administering, to a subject in need thereof, an Fe³⁺ chelator, a Zn²⁺ chelator, an Ni²⁺ chelator, or a combination thereof.

7. The method of claim 6, wherein the chelator is N,N,N′,N′-tetrakis(2-pyridylmethyl)-ethylenediamine (TPEN), DPESA, TPESA, ethylenediaminetetraacetic acid (EDTA), ethylene glycol-bis(2-aminoethylether)-N,N,N′,N′-tetraacetic acid (EGTA), 1,2-bis(o-aminophenoxy)ethane-N,N,N′,N′-tetraacetic acid (BAPTA), and ethylenediamine-N,N′-diacetic-N,N′-di-p-propionic (EDPA), diethylene triamine pentaacetic acid (DETAPAC), dipyridyl, pyridoxal isonicotinoyl hydrazone (PIH), desferrioxamine (DFO), deferiprone (DFP) or deferasirox (DFS) or a combination thereof.

8. The method of claim 1, further comprising an antiviral agent selected from the group consisting of abacavir, aciclovir, acyclovir, adefovir, amantadine, amprenavir, arbidol, atazanavir, atripla, brivudine, cidofovir, combivir, darunavir, delavirdine, didanosine, docosanol, edoxudine, efavirenz, emtricitabine, enfuvirtide, entecavir, entry inhibitors, famciclovir, fixed dose combinations, fomivirsen, fosamprenavir, foscarnet, fosfonet, fusion inhibitors, ganciclovir, gardasil, ibacitabine, imunovir, idoxuridine, imiquimod, indinavir, inosine, integrase inhibitors, interferon type III, interferon type II, interferon type I, interferon, lamivudine, lopinavir, loviride, MK-0518, maraviroc, moroxydine, nelfinavir, nevirapine, nexavir, nucleoside analogues, oseltamivir, penciclovir, peramivir, pleconaril, podophyllotoxin, protease inhibitors, reverse transcriptase inhibitors, ribavirin, rimantadine, ritonavir, saquinavir, stavudine, synergistic enhancers, tenofovir, tenofovir disoproxil, tipranavir, trifluridine, trizivir, tromantadine, truvada, valaciclovir, valganciclovir, vicriviroc, vidarabine, viramidine, zalcitabine, zanamivir, and zidovudine.

9. A method for reducing iron, nickel or zinc toxicity in a biological system, comprising contacting said biological system with a composition comprising a glutathione precursor and a selenium source.

10. The method of claim 9, wherein the glutathione precursor comprises glycine, L-cystine and a glutamate source.

11. The method of claim 9, wherein the selenium source is selenocysteine or selenomethionine.

12. The method of claim 9, wherein the composition further comprises a metallothioneine or a fragment thereof.

13. The method of claim 9, wherein the biological system is a cellular system, a tissue system, an organ system, or an organism.

14. The method of claim 9, wherein the toxicity is due to dysregulated iron, nickel or zinc homeostasis.

15. The method of claim 9, further comprising administering, to a subject in need thereof, a metal chelator.

16. The method of claim 9, further comprising administering, to a subject in need thereof, an Fe³⁺ chelator, a Zn²⁺ chelator, an Ni²⁺ chelator, or a combination thereof.

17. The method of claim 9, wherein the chelator is N,N,N′,N′-tetrakis(2-pyridylmethyl)-ethylenediamine (TPEN), DPESA, TPESA, ethylenediaminetetraacetic acid (EDTA), ethylene glycol-bis(2-aminoethylether)-N,N,N′,N′-tetraacetic acid (EGTA), 1,2-bis(o-aminophenoxy)ethane-N,N,N′,N′-tetraacetic acid (BAPTA), and ethylenediamine-N,N′-diacetic-N,N′-di-3-propionic (EDPA), diethylene triamine pentaacetic acid (DETAPAC), dipyridyl, pyridoxal isonicotinoyl hydrazone (PIH), desferrioxamine (DFO), deferiprone (DFP) or deferasirox (DFS) or a combination thereof.

18. The method of claim 9, further comprising administering N-acetylcysteine, vitamin C, vitamin E, α-lipoic acid, folic acid, vitamins B6 and B12, silibinin, resveratrol or a combination thereof.
